(19) United States
(12) Patent Application Publication
(10) Pub. No.: US 2011/0081298 A1
(43) Pub. Date: Apr. 7, 2011

(54) RATIOMETRIC BIOLUMINESCENT SENSOR FOR IMAGINING OXIDATIVE STRESS

(76) Inventors: Andrew Tsourkas, Philadelphia, PA (US); Julie Czupryna, Yorkville, NY (US)

(21) Appl. No.: 12/896,495

(22) Filed: Oct. 1, 2010

Related U.S. Application Data
(60) Provisional application No. 61/247,635, filed on Oct. 1, 2009.

(51) Int. Cl. A61K 49/00; C12Q 1/66

(52) U.S. Cl. 424/9.6; 435/8

(57) ABSTRACT

This invention provides a novel ratiometric bioluminescent sensor and methods of use thereof. The bioluminescent sensor comprises two luminescent proteins that exhibit different characteristics associated a biological condition, and thereby illuminates differently in response to the biological condition. A ratio between the luminescence of the two luminescent proteins indicates a change in the biological condition. The bioluminescent sensor of the invention can be used to image an oxidative stress or its associated conditions, including a programmed cell death.

### CROSS-REFERENCE TO RELATED APPLICATIONS

This application claims priority to U.S. Provisional Application 61/247,635, filed Oct. 1, 2009, which is incorporated by reference herein in its entirety.

### FIELD OF THE INVENTION

The invention relates to a novel ratiometric bioluminescent sensor and methods of use thereof. Specifically, the invention relates to a novel ratiometric bioluminescent sensor for imaging oxidative stress or associated conditions, including programmed cell death.

### BACKGROUND OF THE INVENTION

Reactive oxygen species (ROS, e.g., oxygen ions, free radicals, and peroxide) are formed as a natural byproduct of metabolic processes and have been shown to play a role in normal cell signaling and function. The level of ROS is typically controlled by various antioxidants that are either taken as dietary supplements (e.g. vitamin E and C) or expressed in cells (e.g. glutathione, superoxide dismutase, and catalase). Under many pathologic conditions, however, there can be an imbalance in these processes, causing a dramatic increase in ROS levels. During these states of ‘oxidative stress’, various vital cellular components can be damaged, including DNA, RNA, proteins and lipids. Pathologic conditions that have already been linked to a state of oxidative stress include cancer, atherosclerosis, cystic fibrosis, type-2 diabetes and neurodegenerative disorders such as Parkinson's or Alzheimer's disease. The ability to investigate oxidative stress/ROS via in vivo imaging could evidently provide unique insight into the aforementioned maladies and cellular processes and facilitate ongoing research pursuits in the areas of therapeutic development and evaluation. Bioluminescence may prove to be a particularly promising imaging modality for this task, given its sensitivity, cost-effectiveness, simplicity, high-throughput screening potential and ability to garner temporal information in cells and in live animal models.

A number of activatable fluorescent probes currently exist for detecting ROS. Perhaps, the most widely utilized are the non-fluorescent leuco dyes, i.e. “dehydro” derivatives of fluorescein, rhodamine, and various other dyes that are oxidized back to the parent dye by some ROS. While these probes have been used to detect oxidative activity in cells and tissue, their oxidation may not easily discriminate between the various ROS. Moreover, none of the Leuco dyes or fluorescein derivatives have been used for imaging oxidative stress directly in live animals.

Although luminol and lucigenin have previously been used to report the presence of ROS, a problematic characteristic of these probes is that light emission requires a two-step reaction. For Electron Spin Resonance, a concern is that ROS formed in vivo may not survive long enough to be detected ex vivo.

While it is well established that some chemotherapeutic agents and radiation therapies can generate ROS that lead to cell death, there is also growing evidence that ROS may be involved in the regulation of both apoptotic and non-apoptotic cell death pathways.

Programmed cell death (PCD) plays a crucial role in the maintenance of homeostasis and is involved in a multitude of biological processes, including embryonic development, aging and the immune response. Unregulated PCD can lead to unwarranted cell accumulation or loss, both of which have been implicated in pathologies such as cancer, autoimmune diseases and neurodegenerative disorders. The ability to image PCD in vivo is imperative to the ongoing research pursuits examining these maladies, especially in the areas of therapeutic development and evaluation.

Commercial methods that have been developed to monitor PCD include measuring the extent of DNA fragmentation, measuring cell permeability, and measuring the activity of distinct intracellular proteases (dead-cell proteases) that are released from membrane compromised cells. Other commercial assays that have been widely adopted for determining the extent of cell death, rely on measuring the activity of mitrochondrial succinate-tetrazolium reductase, i.e. MTT and WST-1 assays. Although all of the aforementioned commercial kits clearly have utility in measuring PCD in cell culture, most of them are not appropriate for real-time imaging and/or imaging in animal models of disease. These limitations generally stem from the need for cell lysis, the use of membrane impermeable substrates, and/or the use of green fluorescent dyes, which are generally masked by high autofluorescence in vivo.

Accordingly, there exists a need for improved sensors for imagining oxidative stress and its associated diseases or conditions including programmed cell death.

### SUMMARY OF THE INVENTION

In one embodiment, the invention provides a ratiometric bioluminescent sensor for imaging a biological condition comprising: a first luminescent protein; and a second luminescent protein, wherein said first and second luminescent proteins exhibit different characteristics associated with said biological condition, and thereby illuminates differently in response to said biological condition, and wherein a ratio between the luminescence of said first and said second luminescent proteins indicates a change in said biological condition.

In another embodiment, the invention provides a method for imaging a biological condition comprising: detecting the luminescence of a first luminescent protein; detecting the luminescence of a second luminescent protein; and determining a ratio between the luminescence of said first and second luminescent proteins, wherein said first and second luminescent proteins exhibit different characteristics associated with said biological condition, and thereby illuminates differently in response to said biological condition, and wherein the determined ratio between the luminescence of said first and second luminescent proteins indicates a change in said biological condition.

In another embodiment, the invention provides a method for diagnosis of a biological condition comprising: detecting the luminescence of a first luminescent protein; detecting the luminescence of a second luminescent protein; and determining a ratio between the luminescence of said first and second luminescent proteins, wherein said first and second luminescent proteins exhibit different characteristics associated with said biological condition, and thereby illuminates differently in response to said biological condition, and wherein the determined ratio between the luminescence of said first and second luminescent proteins indicates a change in said biological condition.

In another embodiment, the invention provides a method for monitoring the progression of a biological condition comprising: detecting the luminescence of a first luminescent protein; detecting the luminescence of a second luminescent protein; and determining a ratio between the luminescence of said first and second luminescent proteins, wherein said first and second luminescent proteins exhibit different characteristics associated with said biological condition, and thereby illuminates differently in response to said biological condition, and wherein the determined ratio between the luminescence of said first and second luminescent proteins indicates the progression of said biological condition.

In another embodiment, the invention provides a method for imaging an oxidative stress comprising: detecting the luminescence of a first luminescent protein; detecting the luminescence of a second luminescent protein; and determining a ratio between the luminescence of said first and second luminescent proteins, wherein said first and second luminescent proteins exhibit different characteristics associated with said oxidative stress, and thereby illuminates differently in response to said oxidative stress, and wherein the determined ratio between the luminescence of said first and second luminescent proteins indicates a change of said oxidative stress.

In another embodiment, the invention provides a method for imaging programmed cell death comprising: detecting the luminescence of a first luminescent protein; detecting the luminescence of a second luminescent protein; and determining a ratio between the luminescence of said first and second luminescent proteins, wherein said first and second luminescent proteins exhibit different characteristics associated with said programmed cell death, and thereby illuminates differently in response to said programmed cell death, and wherein the determined ratio between the luminescence of said first and second luminescent proteins indicates the progression of said cell death.

In another embodiment, the invention provides a method for screening drugs to treat a biological condition, said method comprising: detecting the luminescence of a first luminescent protein; detecting the luminescence of a second luminescent protein; and determining a ratio between the luminescence of said first and second luminescent proteins, wherein said first and second luminescent proteins exhibit different characteristics associated with said biological condition, and thereby illuminates differently in response to said biological condition, and wherein the determined ratio between the luminescence of said first and second luminescent proteins indicates a change of said biological condition, thereby screening drugs to treat said biological condition.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE FIGURES

The invention will be better understood from a reading of the following detailed description taken in conjunction with the drawings in which like reference designators are used to designate like elements.

FIG. 1. Mechanisms of protein modification and/or damage mediated by reactive oxygen species.

FIG. 2. The bioluminescent ratio was determined for Hela cells treated with 5 mM H₂O₂ for up to 24 hrs.

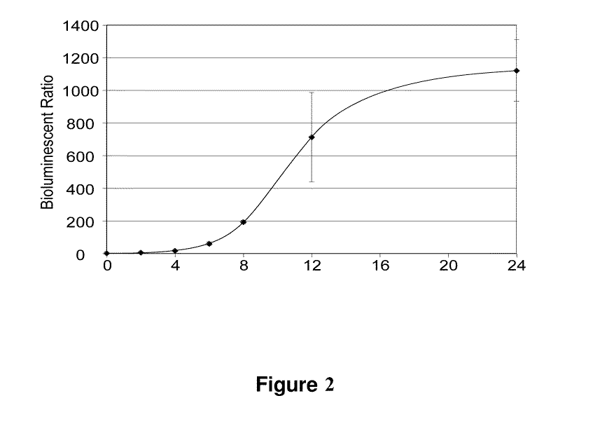

FIG. 3. Response of HeLa-fR cells to STS. HeLa-fR cells were treated with 10 μM STS for 24 hours. (A) Bioluminescent measurements of RLuc8 and fLuc were acquired at various times during the course of treatment. All measurements were normalized to values at 0 hours. The RLuc8:fLuc ratio was subsequently calculated for each time point (right axis). (B) A representative bioluminescent image of RLuc8 and fLuc activity in STS-treated cells is shown as well as the calculated ratiometric image, RLuc8:fLuc. (C) A TUNEL assay for DNA fragmentation was also performed at various times during the course of STS treatment. Analogous bioluminescent and DNA fragmentation measurements were performed on RBS-HeLa cells that were treated with a dosage range of STS for 6 hours. (D) Bioluminescent measurements of RLuc8 and fLuc activity in cells treated with various doses of STS. The RLuc8:fLuc ratio is also shown (right axis). (E) Representative bioluminescent images of RLuc8 and fLuc activity in cells treated with various doses of STS. The calculated ratiometric image of RLuc8:fLuc is also shown. (F) A TUNEL assay for DNA fragmentation was also performed on HeLa-fR cells treated with various doses of STS.

FIG. 4. Assessment of mRNA and protein levels in STS-treated HeLa-fR cells. (A) Normalized RLuc8 and fLuc mRNA levels in HeLa-fR cells treated with PBS (control, C) or 10 μM STS (STS) for 24 hours. mRNA levels were determined by qRT-PCR. (B) Western blot of HeLa-fR cells treated with PBS (−) or 10 μM STS (+) over a time course of 24 hours. β-actin is shown as a loading control.

FIG. 5. Inhibition studies to determine mechanism responsible for activation of the RBS. HeLa-fR cells were pretreated with (A) proteasome inhibitors, (B) protease inhibitors, (C) O₂^{•-} scavengers, (D) ^{•}OH scavengers, or (E) H₂O₂-related scavengers for 1 hour, followed by PBS (white bars) or 10 μM STS for 24 hours (gray bars). The RLuc8:fLuc ratio was calculated and reported. Statistical significance between control (+STS) and individual inhibitors/scavengers (+STS) is indicated with a ** (p<0.01).

FIG. 6. Response of HeLa-fR cells to a hypoxanthine (HX)-xanthine oxidase (XO) reaction and the affect of adenovirus-catalase on STS-treated cells. (A) HeLa-fR cells were subjected to the HX-XO reaction (50 μM HX, 25 mU/mL XO) or PBS (Control) for 24 hours. The RLuc8:fLuc ratio was calculated and reported. (B) A TUNEL assay was used to determine the percent DNA fragmentation, following exposure to HX-XO. (C) HeLa-fR cells were treated with adenovirus-catalase (Adv-Cat) or empty adenovirus (Adv-E) for 24 hours, washed, and incubated in serum-containing media for another 24 hours. Then, 10 μM STS or PBS was added to the cells. After 24 hours the RLuc8:fLuc ratio was calculated and reported. (D) HeLa-fR cells were subjected to the same conditions as in (C) but assayed for H₂O₂ levels using CM-H₂DCFDA.

FIG. 7. Response of purified fLuc and RLuc8 enzymes to H₂O₂. (A) RLuc8 (650 nM) was treated with 0 to 10 mM H₂O₂ for up to 120 minutes. Every 30 minutes, aliquots were removed and subjected to bioluminescence measurements using Dual-Glo Luciferase Assay System (Promega). (B) Analogous studies were performed with fLuc (650 nM). (C) The bioluminescent ratio, RLuc8:fLuc, was calculated for each experimental condition.

FIG. 8. Response of HeLa-fR cells to increasing doses of sodium selenite. Sodium selenite (SSe) was added to HeLa-fR cells at a concentration range of 0-55 μM for 24 hours. (A) Measurements of RLuc8 and fLuc were acquired using the Promega Dual-Glo assay and the RLuc8:fLuc ratios were reported. (B) Intracellular H₂O₂ measurements were obtained using CM-H₂DCFDA. (C) Caspase activity was measured using Caspase-Glo 3/7. (D) The percent DNA fragmentation was determined via a TUNEL assay. In all studies (except TUNEL assays, which provide an absolute measure of cell death), measurements were normalized to values at 0 μM SSe.

FIG. 9. Measurements of RLuc8:fLuc ratio in tumor-bearing mice in vivo, following pretreatment with allopurinol or PBS and subsequent treatment with STS or PBS. Female nude mice bearing HeLa-fR tumor xenografts were imaged prior to and 24 hours after the following inhibitor/drug treatment regiment: (A) PBS/PBS, (B) PBS/STS, (C) Allo/PBS, and (D) Allo/STS. Inhibitors were administered 1 hour prior to drug treatment. (E) Quantitative analysis of the RLuc8:fLuc ratio in live animals. All images were analyzed using Living Image Software. Following background subtraction, the RLuc8:fLuc ratios were calculated and normalized to pretreatment values. Statistical significance: * (p<0.05), ** (p<0.01).

FIG. 10. Analysis of RLuc8:fLuc ratio as a function of time and cell number. (A) For a fixed cell seeding density, the RLuc8 and fLuc bioluminescent signal that was elicited by HeLa-fR cells (PBS-treated) was detected over the course of 24 hrs (left axis) and the RLuc8:fLuc ratio was calculated at each time point (right axis). (B) HeLa-fR cells were plated at various cell densities and the RLuc8:fLuc ratio was measured.

FIG. 11. Caspase 3/7 activity in STS-treated HeLa-fR cells. HeLa-fR cells were treated with 10 μM STS of PBS (untreated) for up to 24 hours. A Caspase-Glo 3/7 assay (Promega) was performed at various times during the course of treatment. All measurements were normalized to values at 0 hours.

FIG. 12. Response of MCF7 and 293T/17 cells to increasing doses of STS. (A-C) MCF7-fR and (D-F) 293T/17-fR cells were treated with a dosage range of STS (0-50 μM) for 6 hours. (A,B) Bioluminescent measurements of RLuc8 and fLuc were acquired for each STS concentration after 6 hours (left axis). The RLuc8:fLuc ratio was subsequently calculated for each STS concentration (right axis). (B,E) A TUNEL assay for DNA fragmentation was performed to provide a measure of cell death. (C,F) Caspase 3/7 activity was determined using a Caspase 3/7 Glo assay (Promega). In all studies (except TUNEL assays, which provide an absolute measure of cell death), measurements were normalized to values at 0 μM STS.

FIG. 13. Response of MCF7 and 293T/17 cells to STS as a function of time. (A-C) MCF7-fR and (D-F) 293T/17-fR cells were treated with 10 μM STS or PBS (untreated) for up to 24 hours. (A,B) Bioluminescent measurements of RLuc8 and fLuc were acquired for at various time points following treatment and the RLuc8:fLuc ratio was calculated and reported. (B,E) A TUNEL assay for DNA fragmentation was performed to provide a measure of cell death. (C,F) Caspase 3/7 activity was determined using a Caspase 3/7 Glo assay (Promega). In all studies (except TUNEL assays, which provide an absolute measure of cell death), measurements were normalized to values at 0 hours.

FIG. 14. Effects of DOX and CPT on HeLa cells. HeLa-fR cells were treated with 1 μM DOX, 10 μM CPT, or PBS (i.e. untreated control) for up to 48 hours. (A) RLuc8 and fLuc bioluminescence measurements were taken at various time points following treatment with DOX and the RLuc8:fLuc ratio was calculated and reported. Measurements of fLuc, RLuc, and the ratio RLuc8:fLuc were also acquired for cells treated with PBS. (B) Measurements of DNA fragmentation and (C) caspase 3/7 activity were also acquired for cells treated with DOX or PBS. (D) Bioluminescent measurements, (E) measurement of DNA fragmentation, and (F) caspase 3/7 activity were also acquired at various times after exposure to CPT or PBS. In all studies (except TUNEL assays, which provide an absolute measure of cell death), measurements were normalized to values at 0 hours.

FIG. 15. Control assays to validate inhibitor effectiveness. HeLa-fR cells were pretreated with either PBS (gray bars) or indicated inhibitors (white bars) for 1 hour before the addition of 10 μM STS. The PBS/Inhibitors and STS were then incubated with the cells for an additional 24 hours. (A) Proteasome inhibitors were assayed using Proteasome-Glo. (B) Protease inhibitors were assayed using Calpain-Glo (Calpain Inhibitor III), CV-Cathepsin B Detection Kit (Pepstatin A) or Caspase-Glo 3/7 (z-vad-fmk). (C) O₂^{•-} scavengers were assayed using DHE. (D) ^{•}OH scavengers were assayed using HPF. (E) H₂O₂-related scavengers were assayed using CM-H₂DCFDA (catalase and allopurinol) or the Cox Activity Assay Kit (aspirin). All measurements were normalized to the mean measurements in the absence of the respective inhibitor. Statistical significance between −inhibitor and +inhibitor is indicated with a * (p<0.05) or ** (p<0.01).

FIG. 16. Dose response of STS-treated cells to H₂O₂-related scavengers/inhibitors. HeLa-fR cells were pretreated with a dosage range of (A) catalase, (B) allopurinol, or (C) aspirin for 1 hour prior to 24 hours of 10 μM STS. The RLuc8:fLuc ratio was calculated and reported. (D) Western blot on HeLa-fR cells pretreated for 1 hour with PBS, 50 U/mL catalase, 100 μM allopurinol or 1 mM aspirin (columns 2-5) before 10 μM STS for 24 hours. β-actin is shown as a loading control.

FIG. 17. Effect of allopurinol pretreatment on STS-treated HeLa cells. HeLa-fR cells were pretreated with either 100 μM allopurinol or PBS for 1 hour prior to the administration of 10 μM STS or PBS. After 24 hours, (A) the RLuc8:fLuc ratio, (B) intracellular H₂O₂ levels as measured by CM-H₂DCFDA, and (C) DNA fragmentation levels, as measured using a TUNEL assay, were recorded for each sample. Statistical significance: *(p<0.05), **(p<0.01).

FIG. 18. Effect of allopurinol pretreatment on STS-treated MCF7 cells. MCF7 cells were pretreated with either 100 μM allopurinol or PBS for 1 hour prior to the administration of 1 μM STS or PBS. After 6 hours, (A) the RLuc8:fLuc ratio, (B) intracellular H₂O₂ levels as measured by CM-H₂DCFDA, and (C) DNA fragmentation levels, as measured using a TUNEL assay, were recorded for each sample. Statistical significance: *(p<0.05), **(p<0.01).

FIG. 19. Effect of allopurinol pretreatment on STS-treated 293T/17 cells. 293T/17 cells were pretreated with either 100 μM allopurinol or PBS for 1 hour prior to the administration of 10 μM STS or PBS. After 24 hours, (A) the RLuc8:fLuc ratio, (B) intracellular H₂O₂ levels, as measured by CM-H₂DCFDA, and (C) percent DNA fragmentation, as measured by a TUNEL assay, were recorded for each sample. Statistical significance: *(p<0.05), **(p<0.01).

FIG. 20. Effect of allopurinol pretreatment on DOX-treated HeLa cells. HeLa-fR cells were pretreated with either 100 μM allopurinol or PBS for 1 hour prior to the administration of 1 μM DOX or PBS. After 48 hours, (A) the RLuc8:fLuc ratio, (B) intracellular H₂O₂ levels, as measured by CM-H₂DCFDA, and (C) percent DNA fragmentation, as measured by a TUNEL assay, were recorded for each sample. Statistical significance: *(p<0.05), **(p<0.01).

FIG. 21. Effect of allopurinol pretreatment on Cpt-treated HeLa cells. HeLa-fR cells were pretreated with either 100 μM allopurinol or PBS for 1 hour prior to the administration of 10 μM Cpt or PBS. After 48 hours, (A) the RLuc8:fLuc ratio, (B) intracellular H₂O₂ levels, as measured by CM-H₂DCFDA, and (C) percent DNA fragmentation, as measured by a TUNEL assay, were recorded for each sample. Statistical significance: *(p<0.05), **(p<0.01).

FIG. 22. SDS-PAGE of purified RLuc8 and fLuc enzymes following exposure to H₂O₂. RLuc8 (650 nM) and fLuc (650 nM) were treated with 0 to 10 mM H₂O₂ for up to 120 minutes. Every 30 minutes, aliquots were removed and analyzed by SDS-PAGE. For all panels: Lane 1: fLuc, Lane 2: RLuc8.

FIG. 23. Protein carbonylation of purified RLuc8 and fLuc enzymes following exposure to H₂O₂. RLuc8 (650 nM) and fLuc (650 nM) were treated with 5 mM H₂O₂ or PBS for 120 minutes. Samples were then assayed for carbonylation using an OxyBlot Protein Carbonylation Detection Kit (Millipore).

FIG. 24. Effect of allopurinol pretreatment on SSe-treated HeLa cells. HeLa-fR cells were pretreated with either 100 μM allopurinol or PBS for 1 hour prior to the administration of 55 μM SSe or PBS. After 24 hours, (A) the RLuc8:fLuc ratio, (B) intracellular H₂O₂ levels as measured by CM-H₂DCFDA, and (C) DNA fragmentation levels, as measured using a TUNEL assay, were recorded for each sample. Statistical significance: *(p<0.05), **(p<0.01).

FIG. 25. Response of MCF7 cells to increasing doses of sodium selenite. Sodium selenite (SSe) was added to MCF7-fR cells at a concentration range of 0-55 μM for 24 hours. (A) Measurements of RLuc9 and fLuc were acquired using the Promega Dual-Glo assay and the RLuc8:fLuc ratios were reported. (B) Intracellular H₂O₂ measurements were obtained using CM-H₂DCFDA. (C) Caspase activity in was measured using Caspase-Glo 3/7. (D) The percent DNA fragmentation was determined via a TUNEL assay. In all studies (except TUNEL assays, which provide an absolute measure of cell death), measurements were normalized to values at 0 μM SSe.

FIG. 26. Effect of allopurinol pretreatment on SSe-treated MCF7 cells. MCF7 cells were pretreated with either 100 μM allopurinol or PBS for 1 hour prior to the administration of 55 μM SSe or PBS. After 48 hours, (A) the RLuc8:fLuc ratio, (B) intracellular H₂O₂ levels as measured by CM-H₂DCFDA, and (C) DNA fragmentation levels, as measured using a TUNEL assay, were recorded for each sample. Statistical significance: *(p<0.05), **(p<0.01).

FIG. 27. Response of MCF7 cells to increasing doses of Resveratrol. Resveratrol (Res) was added to MCF7-fR cells at a concentration range of 0-1000 μM for 24 hours. (A) Measurements of RLuc9 and fLuc were acquired using the Promega Dual-Glo assay and the RLuc8:fLuc ratios were reported. (B) Intracellular H₂O₂ measurements were obtained using CM-H₂DCFDA. (C) Caspase activity in was measured using Caspase-Glo 3/7. (D) The percent DNA fragmentation was determined via a TUNEL assay. In all studies (except TUNEL assays, which provide an absolute measure of cell death), measurements were normalized to values at 0 μM Res.

FIG. 28. Effect of allopurinol pretreatment on Res-treated MCF7 cells. MCF7 cells were pretreated with either 100 μM allopurinol or PBS for 1 hour prior to the administration of 1 mM Res or PBS. After 24 hours, (A) the RLuc8:fLuc ratio, (B) intracellular H₂O₂ levels as measured by CM-H₂DCFDA, and (C) DNA fragmentation levels, as measured using a TUNEL assay, were recorded for each sample. Statistical significance: *(p<0.05), **(p<0.01).

### DETAILED DESCRIPTION OF THE INVENTION

The invention relates to a novel ratiometric bioluminescent sensor and methods of use thereof. Specifically, the invention relates to a novel ratiometric bioluminescent sensor for imaging an oxidative stress or its associated conditions, including a programmed cell death.

In one embodiment, provided herein is a ratiometric bioluminescent sensor for imaging a biological condition comprising: a first luminescent protein; and a second luminescent protein; wherein said first and second luminescent proteins exhibit different characteristics associated with said biological condition, and thereby illuminates differently in response to said biological condition, and wherein a ratio between the luminescence of said first and said second luminescent proteins indicates a change in said biological condition.

In another embodiment, provided herein is a method for imaging a biological condition comprising: detecting the luminescence of a first luminescent protein; detecting the luminescence of a second luminescent protein; and determining a ratio between the luminescence of said first and said second luminescent proteins, wherein said first and second luminescent proteins exhibit different characteristics associated with said biological condition, and thereby illuminates differently in response to said biological condition, and wherein the determined ratio between the luminescence of said first and said second luminescent proteins indicates a change in said biological condition.

In another embodiment, provided herein is a method for diagnosis of a biological condition in a subject comprising: detecting the luminescence of a first luminescent protein; detecting the luminescence of a second luminescent protein; and determining a ratio between the luminescence of said first and said second luminescent proteins, wherein said first and second luminescent proteins exhibit different characteristics associated with said biological condition, and thereby illuminates differently in response to said biological condition, and wherein the determined ratio between the luminescence of said first and said second luminescent proteins indicates a change in said biological condition.

In another embodiment, provided herein is a method for monitoring the progression of a biological condition comprising: detecting the luminescence of a first luminescent protein; detecting the luminescence of a second luminescent protein; and determining a ratio between the luminescence of said first and said second luminescent proteins, wherein said first and second luminescent proteins exhibit different characteristics associated with said biological condition, and thereby illuminates differently in response to said biological condition, and wherein the determined ratio between the luminescence of said first and said second luminescent proteins indicates the progression of said biological condition.

In another embodiment, provided herein is a method for imaging an oxidative stress comprising: detecting the luminescence of a first luminescent protein; detecting the luminescence of a second luminescent protein; and determining a ratio between the luminescence of said first and said second luminescent proteins, wherein said first and second luminescent proteins exhibit different characteristics associated with said oxidative stress, and thereby illuminates differently in response to said oxidative stress, and wherein the determined ratio between the luminescence of said first and said second luminescent proteins indicates a change of said oxidative stress.

In another embodiment, provided herein is a method for imaging a programmed cell death comprising: detecting the luminescence of a first luminescent protein; detecting the luminescence of a second luminescent protein; and determining a ratio between the luminescence of said first and said second luminescent proteins, wherein said first and second luminescent proteins exhibit different characteristics associated with said programmed cell death, and thereby illuminates differently in response to said programmed cell death, and wherein the determined ratio between the luminescence of said first and said second luminescent proteins indicates the progression of said cell death.

In another embodiment, provided herein is a method for screening drugs to treat a biological condition, said method comprising: detecting the luminescence of a first luminescent protein; detecting the luminescence of a second luminescent protein; and determining a ratio between the luminescence of said first and said second luminescent proteins, wherein said first and second luminescent proteins exhibit different characteristics associated with said biological condition, and thereby illuminates differently in response to said biological condition, and wherein the determined ratio between the luminescence of said first and said second luminescent proteins indicates a change of said biological condition, thereby screening drugs to treat said biological condition.

In one embodiment, the first and second luminescent proteins exhibit different characteristics associated with a biological condition, and thereby illuminate differently in response to the biological condition. The differences in illumination may be on a time-scale, a space-scale, or combination thereof. In one embodiment, the luminescent proteins illuminate similarly until a time point, but differently after the time point because of their differences in characteristics. For example, the first and second luminescent proteins may have different half life, and thus the first luminescent protein may degrade at one time and the second luminescent protein may degrade at another time in response to the biological condition.

In another embodiment, the first luminescent protein catalyzes one substrate and the second luminescent protein catalyzes another substrate in response to the biological condition, thus, they illuminate differently based on their substrate catalyzing properties. Other differences in luminescent protein characteristics known to one of skilled in art can also be used as long as they are associated with the biological condition and exhibit their different characteristics in response to the biological condition.

In one embodiment, the first luminescent protein is a first luciferase protein and the second luminescent protein is a second luciferase protein. In one embodiment, the first luciferase protein is a wild-type firefly luciferase (e.g., fLuc). In another embodiment, the first luciferase protein is wild-type Renilla luciferase (i.e. RLuc). In another embodiment, the first luciferase protein is Gaussia. In another embodiment, the first luciferase protein is Chroma Luc CBG68luc. In another embodiment, the first luciferase protein is Chroma Luc CBRluc. In another embodiment, the first luciferase protein is Metridia.

In one embodiment, the second luciferase protein is a mutant firefly luciferase (e.g., fLuc5). In some embodiments, the mutant firefly luciferase comprise the mutation F14R, L35Q, V182K, I232K, F465R, or combination thereof. In one embodiment, the second luciferase protein is RLuc8. In another embodiment, the second luciferase protein is RLuc8.6-535.

In some embodiments, a substrate of the first luciferase protein is luciferin and a substrate of the second luciferase protein is coelenterazine. Other substrates known to one of skilled in the art are also within the scope of the invention.

In a particular embodiment, the first luciferase protein is a wild-type firefly luciferase (fLuc), which has a reported half life of about 3 hrs in non-apoptopic cells, and the second luciferase protein is a stable variant of Renilla Luciferase (e.g., RLuc8), which has a reported serum half life of 130 hrs. fLuc and RLuc8 catalyze unique substrates (e.g., luciferin and coelenterazine, respectively) and exhibit unique bioluminescent emission peaks. The inventors of the instant application have discovered that when cells undergo cell death, the associated increase in oxidative stress causes a rapid increase in bioluminescent ratio (RLuc8 activity:fLuc activity). As a result, the ratio (RLuc8 activity:fLuc activity) is a reliable indicator of oxidative stress and conditions associated with oxidative stress, e.g. cell death.

In one embodiment, luciferase proteins of the invention (e.g., the first and second luciferase proteins) are expressed within a cell by a nucleic acid construct encoding luciferase genes operatively linked to a regulatory sequence, such as a promoter.

In some embodiments of all aspects described herein, any form of luciferase is useful in the methods and assays as disclosed herein, and includes fragments, variants and recombinant forms of luciferase proteins.

In another embodiment, a nucleic acid construct having sequence encoding luciferase proteins (e.g., the first and second luciferase proteins) is introduced into cells by means of a vector. In another embodiment, the vector is a viral vector. One can use any vector commonly known by one of ordinary skill in the art for use to deliver the nucleic acid construct to a cell. In some embodiments, vectors which can be used include, but are not limited to a lentivirus vector, a retroviral vector, a herpes simplex viral vector, an adenovirus vector, an adeno-associated virus (AAV) vector, an EPV vector, an EBV vector and a bacteriophage. In some embodiments, the nucleic acid sequence for luciferase is codon optimized for mammalian gene expression, for example for expression in human cells, for example the luciferase is humanized is humanized luciferase (hLuc).

In one embodiment, the nucleic acid constructs can be delivered by a vector can be taken up actively by cells in vivo following intravenous injection, e.g., hydrodynamic injection. In some embodiments, the nucleic acid constructs can be delivered without the use of a vector, such as delivery of naked nucleic acid by methods commonly known to one of skilled in the art.

In some embodiments, the nucleic acid construct as disclosed herein is introduced into cells from any species and any tissue. In some embodiment, the cells are present in a subject (i.e. the cells are in vivo). In alternative embodiments, the cells are ex vivo, for example the cells can be removed from the subject and optionally cultured or maintained in a conventional culture medium under suitable conditions permitting growth of the cells, and re-introduced into the subject. For example, the cell is cultured in standard tissue culture media containing the necessary reagents to select for cells which stably retain the nucleic acid construct described above. Cells may be cultured in standard tissue culture dishes e.g. multidishes and microwell plates, or in other vessels, as desired. In some configurations, the assay can be conducted in a 96 well; 386-well or other multi-well plates.

The nucleic acid constructs as disclosed herein comprising a nucleic acid encoding a luciferase protein operatively linked to the regulatory sequence, can be introduced simultaneously, or consecutively, each with the same or different markers. Another aspect of the present invention relates to a vector comprising the nucleic acid construct as disclosed herein, such as a vector comprising nucleic acid encoding luciferase proteins (e.g., the first and second luciferase proteins) operatively linked to a regulatory sequence. Another aspect of the present invention relates to a cell comprising such a vector, for example a mammalian cell. In one embodiment, the cell is cancer cell line, for example, but not limited to DU145, Lncap, MCF-7, MDA-MB-438, PC3, T47D, THP1, U87, H460, HL-60, L929, K562, 293, and Saos2.

In some embodiments, the luminescent proteins of the invention may be operably linked to a molecule of interest in order to image the molecule of interest. Examples of the molecule of interest include, but are not limited to, a small molecule compound, a peptide, a protein, a DNA sequence, and an oligo-nucleotide.

One aspect of the present invention relies on the fact that the cells with the introduced nucleic acid constructs as disclosed herein are capable of expressing the luciferase proteins; comprising steps of gene transcription, translation and post-translational modification. The expressed luciferase proteins may be secreted out of the cell, and in some embodiments into biological samples such as the blood. In some embodiments, the kidney filters the secreted luciferase proteins from the blood and it is expelled from the body in waste biological samples, such as urine biological samples.

The nucleic acid constructs as disclosed herein can be used to monitor the progression of a biological condition, for example the progression of an apoptosis or oxidative stress in a subject. The nucleic acid constructs as disclosed herein can be administered to cells in vivo, or in alternative embodiments to cells in culture, in vivo to cells in the body, or ex vivo to cells outside of a subject that can later be returned to the body of the same subject or another. Such cells can be disaggregated or provided as solid tissue.

The compositions comprising the nucleic acid constructs as disclosed herein can be administered as a formulation adapted for passage through the blood-brain barrier or direct contact with the endothelium. In some embodiments, the compositions may be administered as a formulation adapted for systemic delivery. In some embodiments, the compositions can be administered as a formulation adapted for delivery to specific organs, for example but not limited to the liver, bone marrow, or systemic delivery.

Alternatively, the compositions can be added to the culture medium of cells ex vivo. In addition to the active compound, such compositions can contain pharmaceutically-acceptable carriers and other ingredients known to facilitate administration and/or enhance uptake (e.g., saline, dimethyl sulfoxide, lipid, polymer, affinity-based cell specific-targeting systems). The composition can be incorporated in a gel, sponge, or other permeable matrix (e.g., formed as pellets or a disk) and placed in proximity to the endothelium for sustained, local release. The composition can be administered in a single dose or in multiple doses which are administered at different times. The compositions of the invention can be administered by any suitable route known to one of skilled in the art

The expression of the luciferase proteins present in biological samples from the subject can be assessed by bioluminescence. In one embodiment, bioluminescence is used as a measure of expression of the secreted luciferase protein in the transfected cells, and its subsequent secretion into the subjects biological samples. In one embodiment, a luciferin, for example but not limited to, coelenterazine and its analogues is added to the biological sample from the subject, and the bioluminescence is monitored. In another embodiment, biological samples are collected at different time intervals, and optionally stored at +4° C. or −20° C. for a period of time and the subsequently assayed for bioluminescence. In such an embodiment, biological samples can be obtained from the subject at multiple time points, for example during a particular treatment regimen, and assessed for luciferase activity as in indicator to the disease progression or effectiveness of the treatment regime.

In some embodiments, further analysis and monitoring of the progression of a biological condition (e.g., a disease progression) in the subject can be done by taking biological samples from the subject at specific time points in a treatment regimen, for example, before treatment, during treatment and after treatment, for example to monitor tumor regression during treatment. In another embodiment, a subject can be directly monitored in vivo without the need for biological samples.

In one embodiment, a subject can be monitored for progression of a biological condition until the bioluminescence signal is almost or preferable completely eliminated. Accordingly, the present invention provides methods and constructs for a much faster, real time, non-invasive, easy and highly convenient way to monitor progression of a biological condition such as oxidative stress and programmed cell death in a subject.

In another embodiment, the invention provides a kit. The kit may include a set of reagents required to conduct a bioluminescent reaction. In one embodiment, the kit may include the specific luciferases, luciferin and other substrates, solvents and other reagents that may be required to complete a bioluminescent reaction. The kit may include any set of reagents that, under appropriate reaction conditions, yield bioluminescence. Appropriate reaction conditions refers to the conditions necessary for a bioluminescence reaction to occur, such as pH, salt concentrations and temperature. In another embodiment, the kit includes a bioluminescence substrate, luciferin, and two luminescent proteins, which includes enzymes luciferases and photoproteins, and one or more activators. A specific bioluminescence system may be identified by reference to the specific organism from which the luciferase derives. This kit may also include the particular activators necessary to complete the bioluminescence reaction, such as oxygen and a substrate with which the luciferase reacts in the presence of the oxygen to produce light.

In one embodiment, the kits comprises the compositions, e.g., the pharmaceutical compositions, nucleic acids, expression cassettes, vectors, cells of the invention, to image a biological condition. The kits also can contain instructional material teaching methodologies, e.g., how and when to administer the pharmaceutical compositions, how to apply the compositions and methods of the invention to imaging systems. Kits containing pharmaceutical preparations (e.g., chimeric polypeptides, expression cassettes, vectors, nucleic acids) can include directions as to indications, dosages, routes and methods of administration, and the like.

Methods to measure bioluminescence are well known to those skilled in the art. Bioluminescence reactions are also well-known to those skilled in the art, and any such reaction may be adapted for used in combination with articles of manufacture as described herein. In another embodiment, bioluminescence can be measured by Bioluminescence Resonance Energy Transfer (BRET) (Boute et al, (2002) Trends. Pharmacol. Sci. 23; 351-354; Morin & Hastings (1971) J Physiol 77; 313-18). In one embodiment, a green bioluminescence emission can be observed in vivo, which is the result of the luciferase non-radioactively transferring energy to an accessory green fluorescent protein (GFP). Energy transfer between two fluorescent proteins (FRET) as a physiological reporter has been reported (Miyawaki et al, 1997, Natuew, 388; 882-7). A similar reporter is possible with a luciferase-GFP pair (see, e.g. U.S. Pat. No. 6,436,682 which is incorporated in its entirety herein for reference).

In some embodiments, methods to measure bioluminescence are commonly known by persons of ordinary skill in the art and include, for example but not limited to measuring the bioluminescence by detecting luciferase using a microplate luminometer, a CCD imaging system, a luminometer, a photon counter, a gamma counter, a photodiode, or bioluminescence imaging (BLI) instrument. In some embodiments, the methods as disclosed herein further comprise measuring an additional signal from the cell, wherein the additional signal measures the signal from the marker gene to identify the cells containing the vector.

As described herein, the luminescent proteins can be used to image a biological condition. In one embodiment, the biological condition is an oxidative stress. In one embodiment, the first and second luminescent proteins illuminates differently in response to Reactive Oxygen Species (ROS) during an oxidative stress. In another embodiment, the biological condition is an oxidative stress associated disease or condition. Examples of oxidative stress associated disease or condition include, but are not limited to programmed cell death, cancer, atherosclerosis, cystic fibrosis, type-2 diabetes, Parkinson's disease, Alzheimer's disease, and autoimmune disease. In one embodiment, the programmed cell death is an apoptotic cell death. In another embodiment, the programmed cell death is a non-apoptotic cell death.

In another embodiment, the invention provides a method for imaging oxidative stress in a subject comprising: detecting the luminescence of a first luminescent protein; detecting the luminescence of a second luminescent protein; and determining a ratio between the luminescence of said first and said second luminescent proteins, wherein said first and second luminescent proteins exhibit different characteristics associated with said oxidative stress, and thereby illuminates differently in response to said oxidative stress, and wherein the determined ratio between the luminescence of said first and said second luminescent proteins indicates a change of said oxidative stress in said subject.

In another embodiment, the invention provides a method for imaging a programmed cell death in a subject comprising: detecting the luminescence of a first luminescent protein; detecting the luminescence of a second luminescent protein; and determining a ratio between the luminescence of said first and said second luminescent proteins, wherein said first and second luminescent proteins exhibit different characteristics associated with said programmed cell death, and thereby illuminates differently in response to said programmed cell death, and wherein the determined ratio between the luminescence of said first and said second luminescent proteins indicates the progression of said cell death in said subject.

The bioluminescence sensor of the invention can also be used to screen drugs for a biological condition. In one embodiment, a vector containing two luciferases, described herein, can be transgenically delivered to a animal model (e.g., rodents, zebrafish, etc.), and the transgenic animal models can be used to screen a drug for a biological condition. In another embodiment, a vector containing two luciferases, described herein, can be transgenically delivered to a cell (e.g., cancer cell line), and the transgenic cells can be used to screen a drug for a biological condition. Drug screening can also be performed on cells that have been seeded in micro-plates. Accordingly, in another embodiment, the invention provides a method for screening drugs to treat a biological condition, said method comprising: detecting the luminescence of a first luminescent protein; detecting the luminescence of a second luminescent protein; and determining a ratio between the luminescence of said first and said second luminescent proteins, wherein said first and second luminescent proteins exhibit different characteristics associated with said biological condition, and thereby illuminates differently in response to said biological condition, and wherein the determined ratio between the luminescence of said first and said second luminescent proteins indicates a change of said biological condition, thereby screening drugs to treat said biological condition.

In another embodiment, a ratio between the luminescence of the first and second luminescent proteins can be determined by any method known to one of skilled in the art. In some embodiments, the ratio may be statistically evaluated using a suitable statistical method known to one of skilled in the art. In some embodiments, a system or processor coupled to the image detection system can automatically calculate the ratios and provide the results. It should be understood that this invention is not limited to the particular methodology, protocols, etc., described herein and as such can vary. The terminology used herein is for the purpose of describing particular embodiments only, and is not intended to limit the scope of the present invention.

As used herein, “luminescence” refers to the detectable EM radiation, generally, UV, IR or visible EM radiation that is produced when the excited product of an exergic chemical process reverts to its ground state with the emission of light. Chemiluminescence is luminescence that results from a chemical reaction. Bioluminescence is chemiluminescence that results from a chemical reaction using biological molecules, synthetic versions or analogs thereof, as substrates and/or enzymes.

The term “bioluminescence” as used herein is a type of chemiluminescence, refers to the emission of light by biological molecules. Essential conditions for bioluminescence often comprise; molecular oxygen, either bound or free in the presence of an oxygenase; a luciferase, which acts on a substrate (e.g. luciferin). The bioluminescence reaction is an energy-yielding chemical reaction in which a specific chemical substrate, e.g. a luciferin, undergoes oxidation, catalyzed by an enzyme, a luciferase.

The term “bioluminescence sensor” refers to the components that are necessary and sufficient to generate bioluminescence. These include luciferase, substrates and any necessary co-factors or conditions. Virtually any bioluminescent system known to those skilled in the art will be amenable to use in the methods described herein.

The term “cell” used herein refers to any cell, prokaryotic or eukaryotic, including plant, yeast, worm, insect and mammalian. Mammalian cells include, without limitation; primate, human and a cell from any animal of interest, including without limitation; zebrafish, mouse, hamster, rabbit, dog, cat, domestic animals, such as equine, bovine, murine, ovine, canine, feline, etc. The cells may be a wide variety of tissue types without limitation such as; hematopoietic, neural, mesenchymal, cutaneous, mucosal, stromal, muscle spleen, reticuloendothelial, epithelial, endothelial, hepatic, kidney, gastrointestinal, pulmonary, T-cells etc. Stem cells, embryonic stem (ES) cells, ES-derived cells and stem cell progenitors are also included, including without limitation, hematopoeitic, neural, stromal, muscle, cardiovascular, hepatic, pulmonary, gastrointestinal stem cells, etc. Yeast cells may also be used as cells in this invention. The cells may include a cancer cell line, for example, but not limited to DU145, Lncap, MCF-7, MDA-MB-438, PC3, T47D, THP1, U87, H460, HL-60, L929, K562, 293, and Saos2.

As used herein, “subject” refers to any mammal including a human or any other animal. A subject can be a patient, which refers to a human presenting to a medical provider for diagnosis or treatment of a disease. A subject includes pre and post natal forms.

The following examples are presented in order to more fully illustrate the preferred embodiments of the invention. They should in no way be construed, however, as limiting the broad scope of the invention.

### EXAMPLES

### Example 1

### RLuc8 Exhibits Longer Half-Life than Wild-Type fLuc and RLuc in Apoptotic Cells

The experiments demonstrated that the activity of wild-type firefly luciferase (fLuc) is rapidly reduced in apoptotic cells. Specifically, when Hela cells stably expressing wild-type Firefly luciferase were treated with 10 μM staurosporine (STS), the bioluminescent signal was reduced to <40% of its maximum by 8 hours of treatment. Wild-type Renilla luciferase exhibited a similar loss of activity. Conversely, a stable variant of Renilla luciferase, RLuc8, only exhibited a slight loss in signal over the same time period. The relationship between fLuc and RLuc8 activity is presented in here as an increase in the bioluminescent ratio (RLuc8 activity:fLuc activity). No change in the bioluminescent ratio was observed in untreated cells over the same time period, i.e. the bioluminescent ratio remained one. These findings led us to investigate the mechanism responsible for the rapid loss of fLuc activity in apoptotic cells. Caspase-3/7 Glo and a TUNEL assay were used to confirm PCD.

It should be noted that although similar results were obtained in live cells, the above measurements of fLuc and RLuc8 activity were acquired using the in vitro assay kit, Dual-Glo (Promega). Therefore, one could immediately rule out a lack of ATP, Mg²⁺, and oxygen as factors contributing to the loss of fLuc activity in apoptotic cells.

### Example 2

### Differential Sensitivity of Firefly Luciferase and RLuc8 Activity to Oxidative Stress Allows for Detection of Caspase-Dependent and Caspase-Independent Cell Death

When HeLa cells stably expressing Firefly Luciferase (fLuc) were treated with staurosporine (STS), there was a rapid loss in bioluminescence. Extensive inhibition studies that targeted known intracellular protein degradation/modification pathways revealed that reactive oxygen species (ROS), primarily hydrogen peroxide (H₂O₂), were responsible for the loss in fLuc activity. Consistent with these findings, the direct application of H₂O₂ to purified fLuc enzymes led to a dose- and time-dependent reduction in bioluminescence. Comparatively, RLuc8, a stable variant of Renilla luciferase, was far less sensitive to ROS and exhibited a longer-lived signal in apoptotic cells. These findings demonstrated that an increase in the bioluminescent ratio, RLuc8:fLuc, would be indicative of elevated levels of intracellular ROS. Accordingly, elevated levels of H₂O₂ in various cases of caspase-dependent and -independent cell death were consistently reflected by an increase in the bioluminescent ratio. Furthermore, elevated levels of ROS were detected in STS-treated murine tumor models.

### Materials and Methods

### Plasmid Vector Construction

The internal ribosome entry site (IRES) from the pIRES2-DsRed Express Vector (Clontech, Mountain View, Calif., USA) was cloned into the pIRES vector (Clontech) using restriction enzymes NheI and KpnI (New England Biolabs, Ipswich, Mass., USA), creating the pIRES12 vector. The phRL-CMV vector (Promega, Madison, Wis., USA) encoding Renilla Luciferase was modified to contain eight amino acid mutations within the rLuc sequence (A55T, C124A, S130A, K136R, A143M, M185V, M253L, and S287L) using the QuikChange Multi Site Directed Mutagenesis Kit (Stratagene, La Jolla, Calif., USA) according to the manufacturer's instructions, ultimately creating RLuc8. The DNA sequence encoding Firefly Luciferase (fLuc) from the pGL3-Basic vector (Promega) was PCR amplified and inserted into pCDNA3.1+ (Invitrogen, Carlsbad, Calif., USA) between the BamHI and EcoRI restriction sites. The fLuc sequence was subsequently cloned into pIRES12 using the NheI and XbaI restriction sites of fLuc-pcDNA3.1+ and the XbaI site of pIRES12. RLuc8 was cloned into the pIRES12-fLuc vector at the NheI and BglII restriction sites. The resulting Rluc8-IRES-fLuc sequence was cloned into the pLENTI6/V-5 TOPO vector (Invitrogen) per the manufacturer's instructions to create the fLuc-RLuc8 (fR) vector.

### Inhibition Assays

To induce cellular stress, HeLa cells were treated with Staurosporine (STS), hydrogen peroxide, or hypoxanthine and xanthine oxidase for 0 to 24 hours at concentrations indicated in Table 7. Stressed and unstressed cells were also incubated with inhibitors that target various intracellular protein degradation/modification pathways. The specific compounds/proteins that were utilized as inhibitors and the respective final working concentrations are also listed in Table 7. Inhibitors were added to cells 1-hr prior to adding inducers of cellular stress and left in the media for the duration of the treatment. Analogous controls were conducted in the absence of stress inducers.

### PCD Induction

To induce caspase dependent PCD, cells were treated with staurosporine (STS), Doxorubicin (DOX), or camptothecin (CPT) (Sigma, St. Louis, Mo.) in complete medium at indicated concentrations and time points. To induce caspase-independent PCD, cells were treated with sodium selenite (SSe, MP Biomedicals, Solon, Ohio) or resveratrol (Res, Enzo Life Sciences, Plymouth Meeting, Pa.) in complete medium at indicated concentrations and time points. In studies where allopurinol (Allo, MP Biomedicals) is used as an inhibitor, it was added at a final concentration of 100 μM to cells in complete medium for 1 hour prior to the addition of the death-inducing drugs and remained in the medium throughout the study.

### Cellular Bioluminescence Assays

Unless otherwise noted, bioluminescence assays were performed 24 hours after plating 10,000 HeLa-fR cells/well in a white-walled 96 well tissue culture plate (BD Biosciences, Franklin Lakes, N.J., USA). The Dual-Glo Luciferase Assay System (Promega) was utilized according to the manufacturer's instructions to obtain both fLuc and Rluc8 bioluminescence measurements from an Infinite 200 plate reader (Tecan, Mannedorf, Switzerland). In the case of using MnTMPyP as an inhibitor, the cell media was removed, cells were gently washed and the media was replaced before using the Dual-Glo Luciferase Assay System as it was found that the compound interfered with the bioluminescence measurements.

### Results

fLuc, but not RLuc8, Bioluminescence Decreases in Apoptotic Cells, Resulting in an Increase in the RLuc8:fLuc Bioluminescence Ratio

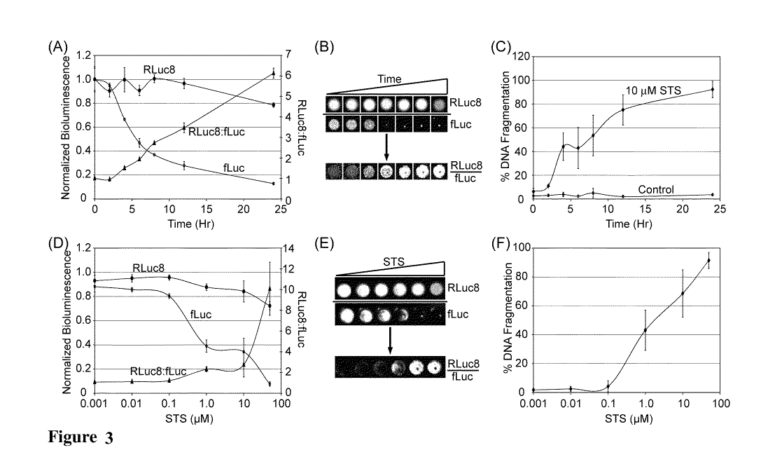

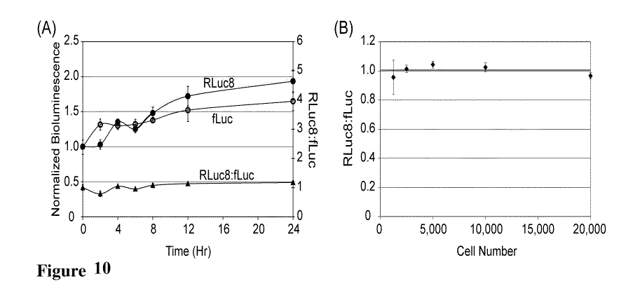

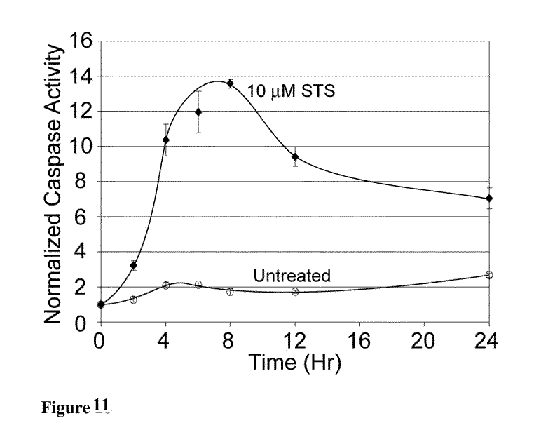

When HeLa cells expressing fLuc and RLuc8 (i.e. HeLa-fR) were treated with 10 μM staurosporine (STS), a drug that induces apoptosis, the bioluminescent signal from fLuc was significantly reduced over a time period of 24 hours, while the signal from Rluc8 remained comparatively stable (FIG. 3A). This can be represented as an increase in the bioluminescent ratio (Rluc8 activity:fLuc activity), as shown on the secondary axis in FIG. 3A. Representative bioluminescent images obtained from HeLa-fR cells under the same conditions are shown in FIG. 3B. Little to no increase in the Rluc8:fLuc ratio was observed in cells that were not treated with STS over the same time period (FIG. 10A). Further, the Rluc8:fLuc ratio was independent of cell number (FIG. 10B). A TUNEL assay confirmed that the STS-treatment was sufficient to induce cell death over the indicated time course (FIG. 3C). A caspase-GLO assay confirmed that cell death was caspase-dependent (FIG. 11).

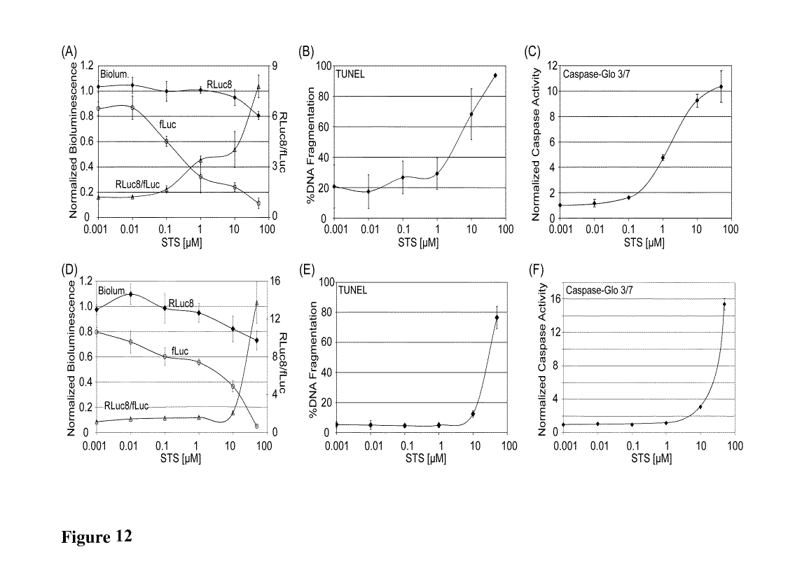

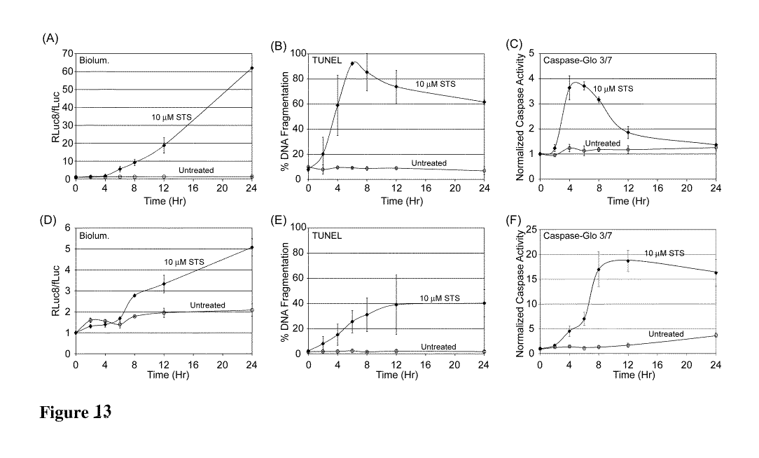

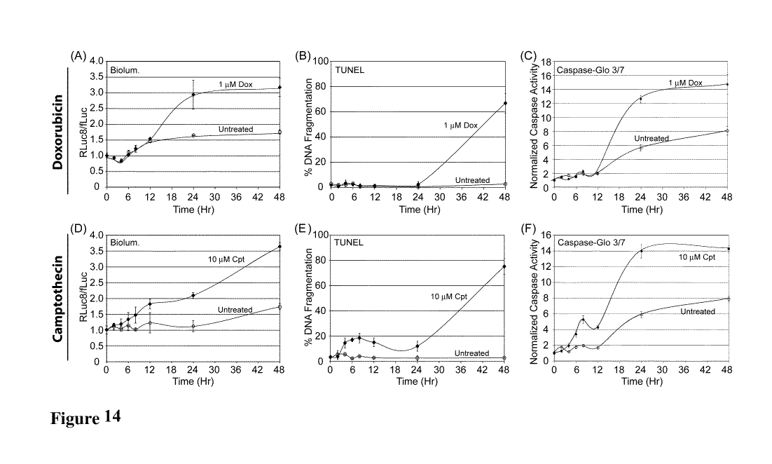

When HeLa-fR cells were treated with increasing doses of STS, the Rluc8:fLuc ratio and extent of cell death also increased in a dose-dependent manner (FIG. 3D-F). Importantly, these findings were not unique to STS-treated HeLa cells. A dose- and time-dependent increase in the RLuc8:fLuc ratio was also observed when 293T/17 cells and MCF-7 cells were treated with STS, as well as when HeLa cells were treated with doxorubicin and camptothecin (FIGS. 12, 13, and 14). These findings show that the preferential loss of fLuc activity during apoptosis, compared with RLuc8, is a common outcome. In each case a caspase-GLO assay confirmed that cell death was caspase-dependent.

Although it was not surprising that RLuc8 exhibited prolonged bioluminescent activity in apoptotic cells, compared with fLuc, it remained unclear which intracellular mechanism(s) was specifically responsible for the loss in fLuc activity. Since all measurements of fLuc and RLuc8 activity that are shown were acquired using the in vitro assay kit, Dual-Glo (Promega), a lack of ATP, Mg²⁺, and oxygen could immediately be ruled out as factors contributing to changes in the Rluc8:fLuc ratio in STS-treated cells. It should be noted that similar bioluminescent measurements were obtained from live and lysed cells, nonetheless.

Protein Levels, but not mRNA Levels Mimic Bioluminescent Data

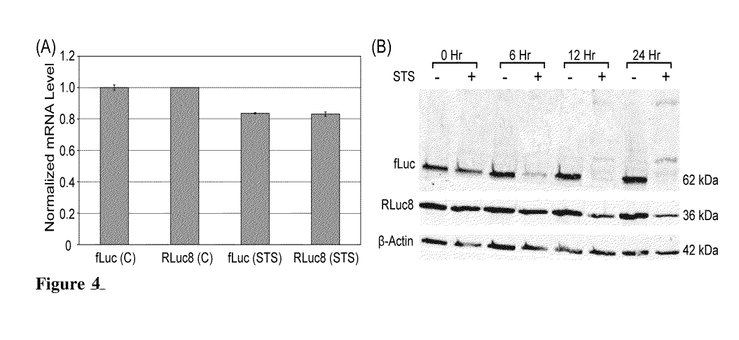

Quantitative RT-PCR and western blot analyses were performed to determine whether changes in RNA or protein levels were responsible for the preferential loss of fLuc activity, compared with RLuc8 activity. Previous studies have shown that oxidative stress can trigger the degradation of both mRNA and proteins. If RNA was degraded, a drastic difference in the relative level of fLuc and Rluc8 mRNA expression would be expected after STS treatment, compared with PBS-treated controls; however, this was not the case. Although slight reductions in fLuc and RLuc8 RNA expression were observed in STS-treated cells, the relative expression remained constant (FIG. 4A).

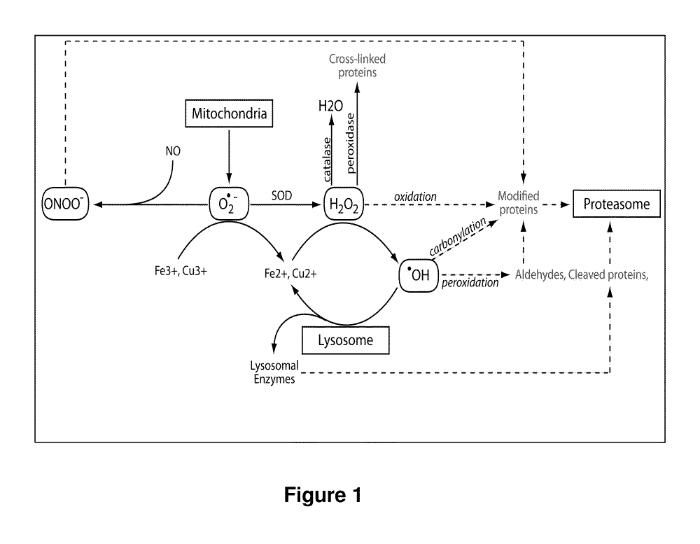

In contrast to RNA expression, western blot analysis revealed that fLuc protein levels decreased over the time course of STS treatment, while Rluc8 protein levels remained relatively stable, mirroring the bioluminescent measurements. Additionally, several higher molecular weight bands were observed after fLuc staining at the later time points, particularly 24 hours, introducing the possibility of protein cross-linking and/or post-translational modification. Based on these findings, we began to investigate various cellular pathways involved in protein degradation and/or modification. FIG. 1 outlines some of the key pathways that were investigated, which included proteasomal and lysosomal pathways, apoptotic pathways, and oxidative stress.

H₂O₂ is Prominently Involved in the Discrepancy Between fLuc and RLuc8 Bioluminescence

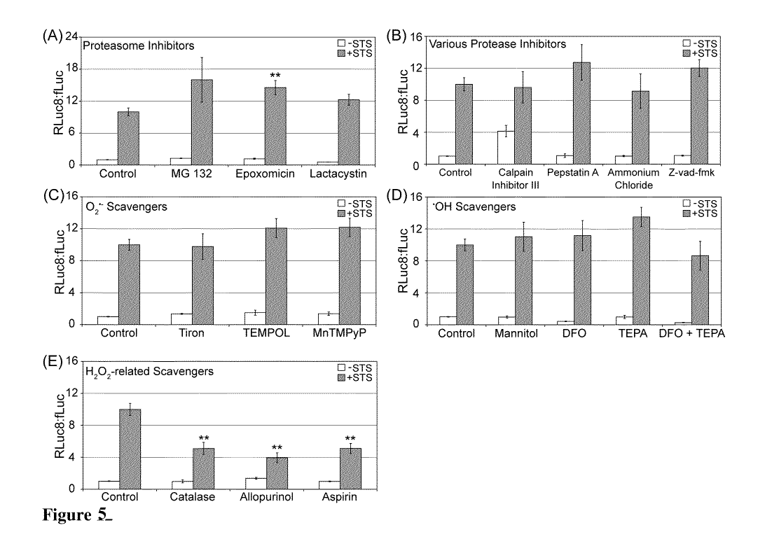

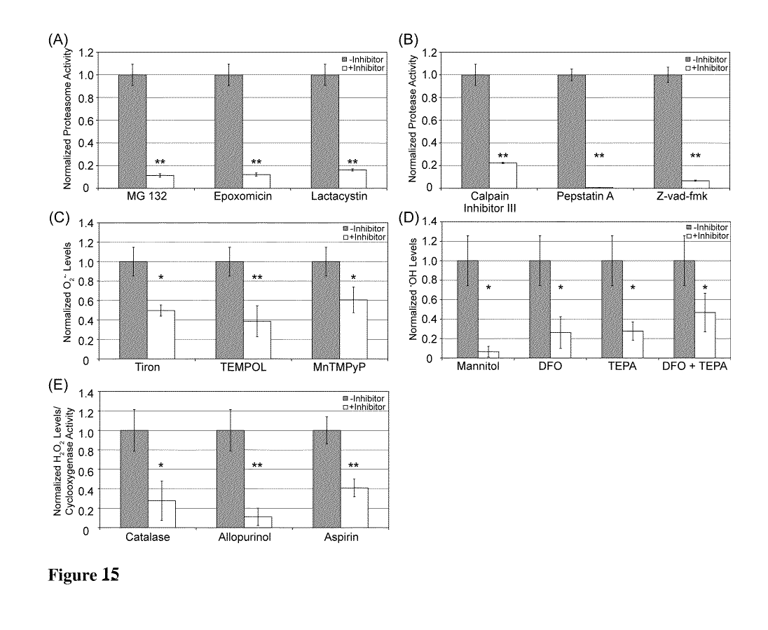

To explicate the root cause responsible for the loss in fLuc activity in STS-treated cells and the corresponding increase in the Rluc8:fLuc ratio, systematic inhibition/scavenger studies were performed to individually silence key pathways known to cause protein degradation/modification. The various inhibitors/scavengers that were tested and their targets are listed in Table 7. It was expected that certain inhibitors would rescue fLuc activity, resulting in a corresponding decrease in the RLuc8:fLuc ratio in STS-treated HeLa-fR cells. Since the proteasome is a prominent source of intracellular protein degradation, it was naturally one of the first molecular entities we investigated for the inhibition studies. Surprisingly, employment of the proteasome inhibitors MG-132, epoxomicin and lactacystin did not rescue fLuc activity and actually led to an increase in the RLuc8:fLuc ratio (FIG. 5A), i.e. fLuc activity was even further reduced relative to RLuc8. The efficacy of the proteasome inhibitors was confirmed by performing analogous studies with Proteasome-GLO (FIG. 15A). These results provided strong evidence that enhanced proteasomal degradation was not responsible for the loss of fLuc activity in STS-treated cells. Since proteasome inhibitors have previously been shown to increase intracellular ROS levels, these findings indicated at ROS as a potential cause for the loss of fLuc activity.

Next, inhibitors for various proteases associated with apoptosis and lysosomal degradation were evaluated, namely calpain Inhibitor III (inhibits calpains), Pepstatin A (inhibits aspartyl proteases), ammonium chloride (inhibits phagosome-lysosome fusion), and z-vad-fmk (pan caspase inhibitor). As shown in FIG. 5B, none of these inhibitors significantly affected the Rluc8:fLuc ratio of STS-treated cells. The efficacy of the various protease inhibitors was confirmed by appropriate commercial assays (FIG. 15B). While the increase in the Rluc8:fLuc ratio observed upon use of the Calpain Inhibitor III on untreated (−STS) cells requires further exploration, one possible explanation may involve the decrease of intracellular antioxidant glutathione (GSH) levels and the associated increase in oxidative stress that has been shown to occur with the addition of this inhibitor.

Once the proteasome and various proteases were shown to have insignificant effects on the mechanism responsible for the increase in the Rluc8:fLuc ratio in STS-treated cells, focus was shifted to three oxygen byproducts associated with oxidative stress, i.e. superoxide (O₂^{•-}), hydroxyl radical (^{•}OH) and hydrogen peroxide (H₂O₂). Three O₂^{•-} scavengers were tested—Tiron, TEMPOL (both cell-permeable O₂^{•-} scavengers) and MnTMPyP (a cell-permeable superoxide dismutase (SOD) mimetic)—for their ability to reduce the Rluc8:fLuc ratio by rescuing fLuc activity. As shown in FIG. 5C, none of the superoxide scavengers significantly reduced the Rluc8:fLuc ratio. This was somewhat expected given the short half-life of O₂^{•-} and its function as a precursor to other oxidizing agents. The efficacy of the O₂^{•-} scavengers was confirmed by performing a dihydroethidium assay (FIG. 15C).

Next, three ^{•}OH scavengers/inhibitors, namely mannitol (specific ^{•}OH scavenger), deferoxamine (DFO, iron chelator) and tetraethylenepentamine (TEPA, copper chelator) were examined. The two metal chelators, alone and in combination, were used to effectively reduce the amounts of metals available for the Fenton reaction (Fe²⁺+H₂O₂→Fe³⁺+^{•}OH+OH⁻). Interestingly, none of the ^{•}OH scavengers/inhibitors provided protection for fLuc activity. Accordingly, these agents did not reduce the Rluc8:fLuc ratio in STS-treated cells (FIG. 5D). It may be argued that the slight increase in Rluc8:fLuc ratio that was observed following the addition of TEPA to the HeLa-fR cells could be a result of excess H₂O₂ buildup from the prevention of the Fenton reaction, however further studies are warranted especially since the combination of DFO+TEPA did not yield an additive effect. The effective reduction of intracellular ^{•}OH following the addition of each scavenger/inhibitor was confirmed by a decrease in hydroxyphenyl fluorescein fluorescence (FIG. 15D).

The final group of inhibitors/scavengers that were examined was associated with reducing intracellular H₂O₂ levels, i.e. catalase (scavenger), allopurinol (xanthine oxidase (XO) inhibitor), and acetylsalicylic acid (aspirin, cyclooxygenase (COX) inhibitor). Catalase converts H₂O₂ to water, allopurinol inhibits XO, an enzyme that catalyzes the oxidation of hypoxanthine and xanthine, creating H₂O₂ as a byproduct, and aspirin inhibits COX enzymes, which possess peroxidase activity. The addition of all three of these reagents resulted in significant reduction (p<0.01) in the Rluc8:fLuc ratio, as see in FIG. 5E. A reduction in intracellular H₂O₂ following the addition of each inhibitor/scavenger was confirmed by a decrease in CM-H₂DCFDA fluorescence (FIG. 15E).

Catalase, Allopurinol and Aspirin Rescue fLuc in a Dose-Dependent Manner and Rescue fLuc Protein Levels in STS Treated Cells

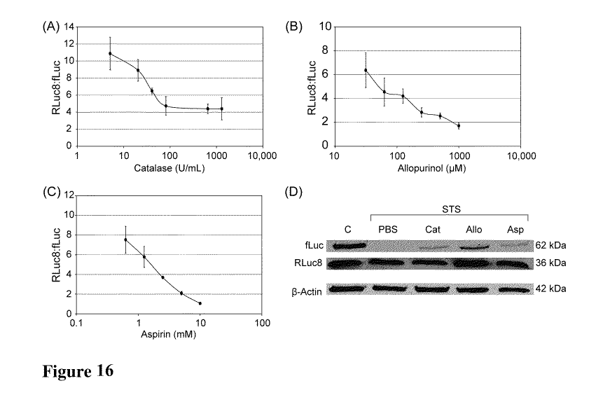

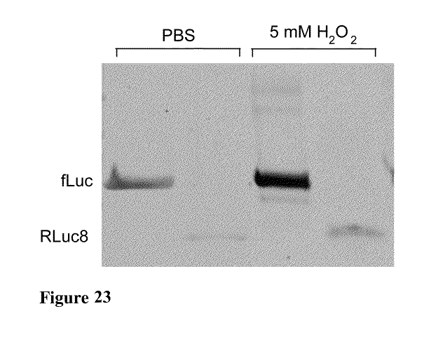

To validate the protective effect of H₂O₂ inhibitors/scavengers on fLuc activity, HeLa-fR cells were pretreated with increasing doses of catalase (FIG. 16A), allopurinol (FIG. 16B) or aspirin (FIG. 16C) for 1 hour prior to the addition of 10 μM STS. After a 24 hr incubation period with the various H₂O₂ inhibitors/scavengers and STS, the bioluminescent ratio was measured. It was found that each inhibitor/scavenger effectively rescued fLuc activity, thus reducing the Rluc8:fLuc ratio, in a dose-dependent manner. A Western blot was also performed (using the same pretreatment conditions from FIG. 5F) to directly determine the effect of the various H₂O₂ inhibitors/scavengers on bioluminescent protein levels. Consistent with the observed recovery in fLuc activity, STS-treated RBS-fR cells that were pretreated with catalase, allopurinol, and aspirin exhibited higher levels of fLuc protein (FIG. 23D, column 3-5) compared to cells treated with STS in the absence of inhibitor (i.e. PBS, column 2).

Allopurinol can Rescue fLuc Activity, Independent of Cell Death

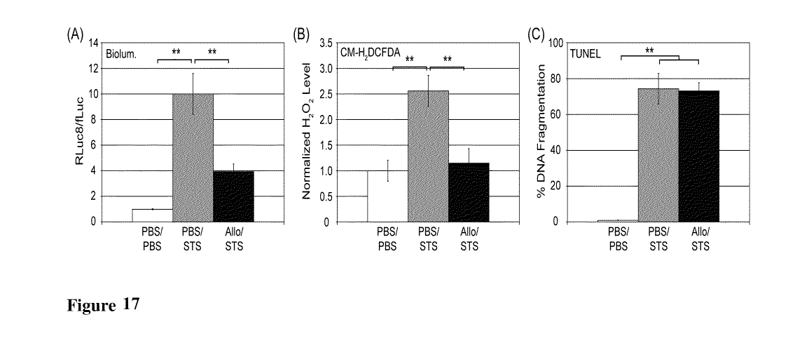

Although pretreating HeLa-fR cells with allopurinol, prior to the addition of STS, helped reduce intracellular H₂O₂ levels and decrease the Rluc8:fLuc ratio by stabilizing fLuc activity, TUNEL assays revealed that allopurinol did not provide protection against cell death (FIG. 17). These findings demonstrate that the processes associated with fLuc inactivation are separate and distinct from cell death, further supporting the notion that the various proteases that are activated/upregulated in apoptotic cells are not responsible for fLuc inactivation. Moreover, considering that allopurinol specifically inhibits xanthine oxidase, a significant source of hydrogen peroxide (and superoxide and hydroxyl radicals to a lesser extent), these findings provide strong evidence that ROS are primarily responsible for fLuc inactivation in apoptotic cells. It is also interesting to note that since significant reductions in the intracellular level of H₂O₂ did not prevent cell death, these results also indicate that H₂O₂ is not essential for the progression of programmed cell death in STS-treated HeLa cells, but rather is a downstream byproduct.

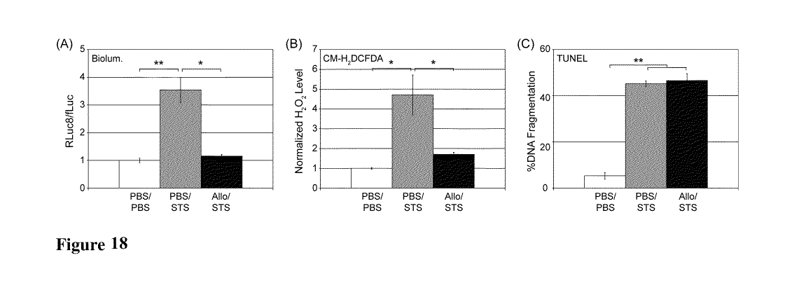

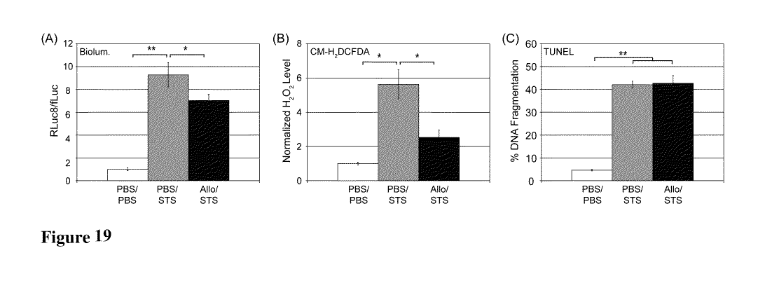

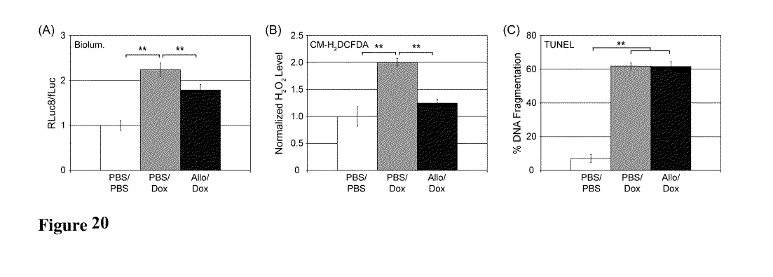

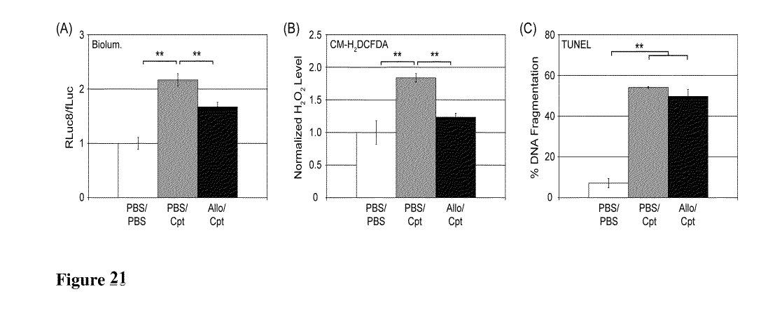

While the results shown thus far specifically pertained to STS-treated HeLa-fR cells, the results are not unique to this cells line or drug. We have found that the protective effect of allopurinol on fLuc is also evident in STS-treated 293T/17 cells and MCF7 cells, as well as HeLa cells treated with camptothecin and doxorubicin (FIG. 18-21). Therefore, the apparent role of ROS in fLuc inactivation seems to be a common phenomenon. Notably, reductions in H₂O₂ did not prevent cell death in any of the studies.

fLuc Activity in Cells is Highly Responsive to H₂O₂

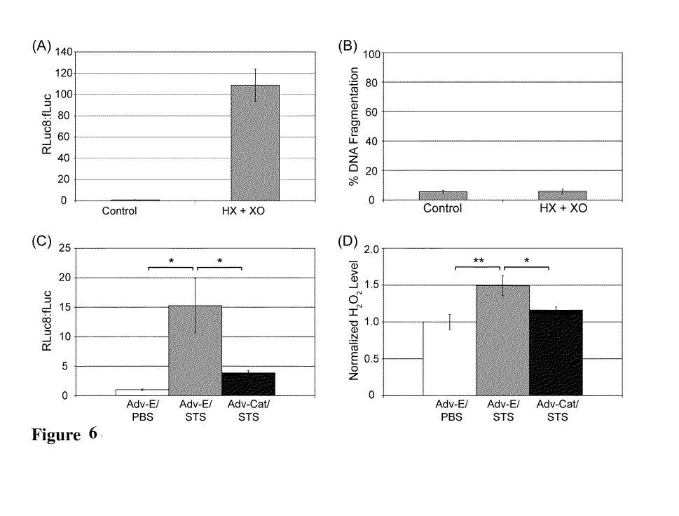

Having shown that various inhibitors/scavengers of H₂O₂ exhibited a stabilizing affect on fLuc activity in apoptotic cells, additional studies were performed that dealt with H₂O₂ more directly. First, we investigated the response of HeLa-fR cells to the hypoxanthine (HX)-XO reaction, which allowed for the continual extracellular production of H₂O₂. After 24 hours, it was found that the bioluminescent ratio increased dramatically, reaching levels that were significantly higher than what was observed previously with STS treatment (FIG. 6A). Additionally, it was found that the HX-XO reaction did not cause cell death, as indicated by a TUNEL assay (FIG. 6B). These results provide strong evidence that the observed loss in fLuc activity is specifically associated with elevated levels of ROS as opposed to other mechanisms that are a consequence of cell death.

Second, to specifically reduce H₂O₂ in apoptotic cells, HeLa-fR cells were infected with an adenoviral vector coding for catalase, prior to STS treatment. Overexpression of catalase led to a dramatic improvement in the stability of fLuc activity in STS-treated HeLa-fR cells and a corresponding decrease in the Rluc8:fLuc ratio, compared with control cells (i.e. cells treated with STS and empty adenovirus) (FIG. 6C). A reduction in intracellular H₂O₂ in cells pretreated with adenovirus-catalase was confirmed by a decrease in CM-H₂DCFDA fluorescence (FIG. 6D).

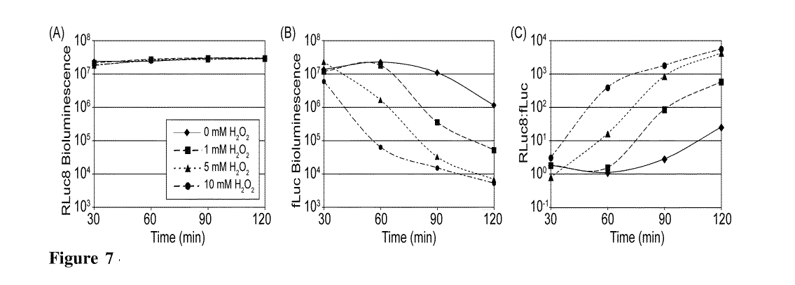

As a final study to assess the responsiveness of fLuc and RLuc8 to H₂O₂, purified enzymes were exposed to increasing concentrations of H₂O₂ (0, 1, 5, and 10 mM). Aliquots from each reaction were removed and analyzed for bioluminescence over the course of 2 hours of treatment. Similar to the cell studies, a dose and time dependent decrease in fLuc bioluminescence was observed, while RLuc8 activity remained stable under the same conditions. The corresponding increase in the RLuc8:fLuc ratio as a function of time and dose is shown in FIG. 7.

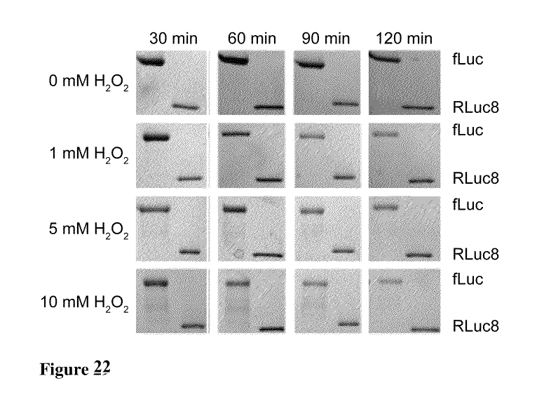

In addition to acquiring bioluminescence measurements, aliquots from each reaction were also subjected to SDS-PAGE (FIG. 22). It was found that while some degradation of fLuc occurred at higher doses of H₂O₂ and at later incubation times, the extent of degradation was less extensive than the loss of bioluminescence. For example, when fLuc was treated with 5 mM H₂O₂ for 90 minutes, the bioluminescent signal was reduced to less than 1% of its starting signal; however, more than 1% of intact protein was still clearly present. These findings show that oxidation of specific amino acids are likely the primary cause for signal loss, not degradation.

fLuc is More Susceptible to Carbonylation than RLuc8 Upon Exposure to H₂O₂

A possible mechanism for fLuc inactivation involves protein carbonylation, as this modification can serve as a trigger for protein degradation. Proteins can become carbonylated directly through oxidative attack on amino acid side chains as well as indirectly through reaction with lipid radicals, carbohydrate radicals and nucleic acid radicals. To determine whether H₂O₂ alone could cause carbonylation of fLuc and/or RLuc8, the purified luciferase enzymes were exposed to H₂O₂ for 2 hours and subsequently assayed for carbonylation using the Oxyblot Protein Carbonylation Detection Kit (FIG. 23). Interestingly, it was found that H₂O₂ led to a dramatic increase in the extent of fLuc carbonylation, while RLuc8 remained generally unaffected. These findings indicate that fLuc is more susceptible to H₂O₂-mediated modifications than RLuc8.

The Differential Sensitivity of RLuc8 and fLuc to Oxidative Stress Allows for the Detection of Caspase Independent Cell Death

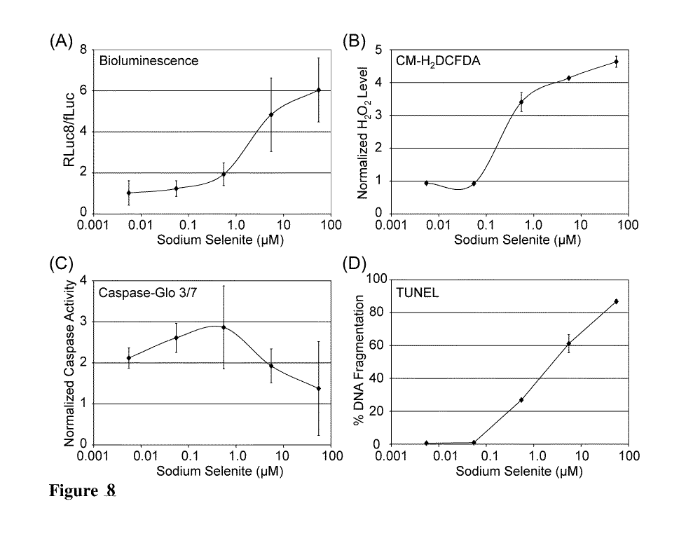

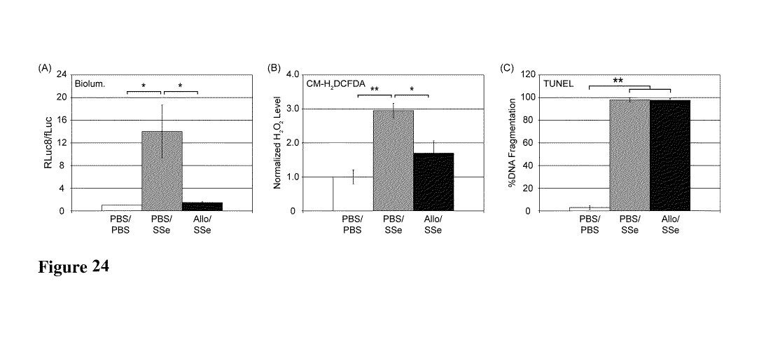

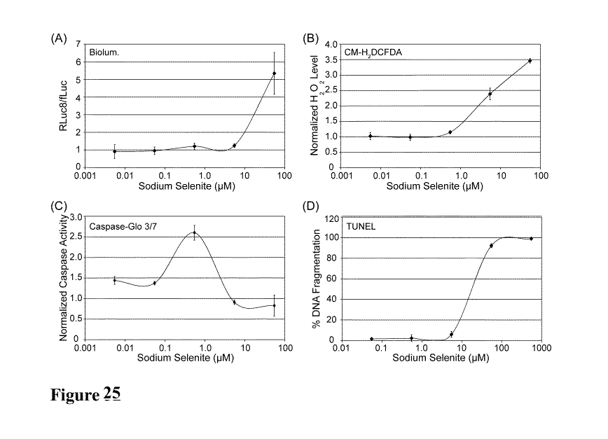

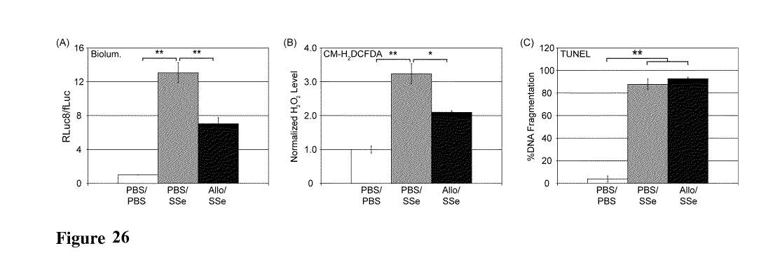

With growing evidence that ROS may be involved in the regulation of various morphologically distinct pathways of cell death, we investigated whether there was an increase in the level of intracellular H₂O₂ in various cases of caspase-independent cell death and whether this could be accurately reflected by an increase in the bioluminescent ratio. First, HeLa cells were treated with sodium selenite (SSe). This compound has been shown to induce caspase-independent PCD in HeLa cells through oxidative stress mediated activation of p53 and p38. When HeLa-fR cells were treated with a dosage range of SSe for 24 hours, the RLuc8:fLuc ratio increased appreciably (FIG. 8A) as did the intracellular levels of H₂O₂ (FIG. 8B); however, caspases 3 and 7 remained relatively inactive (FIG. 8C). Cell death was confirmed using a TUNEL assay to measure DNA fragmentation (FIG. 8D). The increase in DNA fragmentation along with the lack of appreciable caspase activation show that SSe induces caspase-independent PCD and that this form of cell death can be detected by an increase in the RLuc:fLuc ratio. When cells were treated with allopurinol prior to SSe treatment, the intracellular H₂O₂ levels and the RLuc8:fLuc ratio decreased significantly (p<0.05) compared to cells treated with SSe alone (FIG. 24), indicating that the RLuc8:fLuc ratio reflects changes in the intracellular level of H₂O₂. Parallel studies were performed on MCF7-fR cells using SSe, with similar results to the HeLa-fR studies (FIGS. 25 and 26).

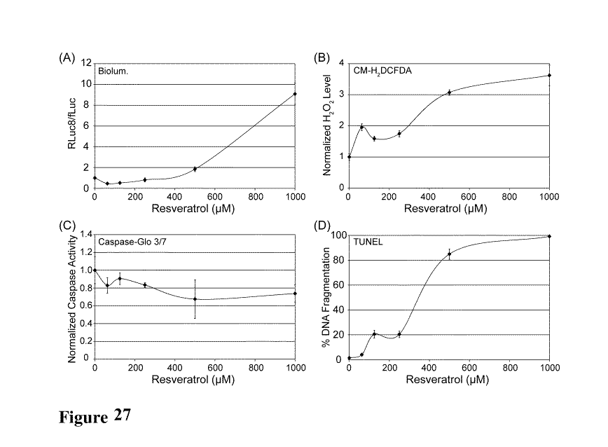

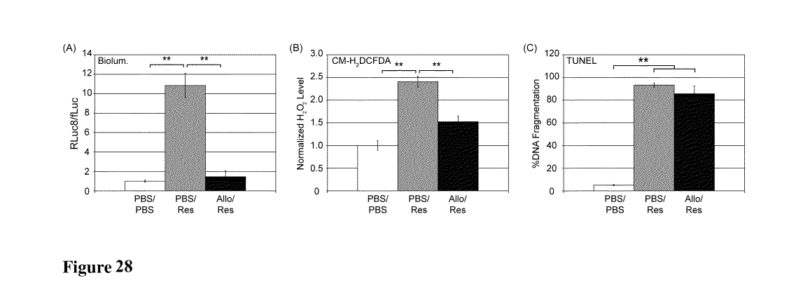

To further examine caspase-independent PCD, resveratrol was used as a cell death inducer for MCF7 cells. Resveratrol has been shown to induce apoptosis through Bcl-2 downregulation, mitochondrial membrane permeabilization and ROS production without caspase 3 activation, PARP cleavage or cytochrome c release. As shown in FIG. 27A, when MCF7-fR cells were treated with increasing dosages of resveratrol for 24 hours, the RLuc8:fLuc ratio increased. Further, caspase 3/7 remained inactive while intracellular H₂O₂ and DNA fragmentation levels increased (FIG. 27B-D). Pretreating MCF7-fR cells with allopurinol prior to administration of resveratrol significantly reduced (p<0.01) the RLuc8:fLuc ratio and H₂O₂ levels, compared to cells treated with resveratrol alone (FIG. 28). These results provide further evidence that H₂O₂ is increased during various morphologically distinct pathways of programmed cell death and that this increase in ROS can be detected by an increase in the RLuc8:fLuc ratio. Given the reported effects of resveratrol on the mitochondria, the measurements of the RLuc8:fLuc ratio may be particularly useful in studies investigating the targeting of this organelle in cancer therapy.

### H₂O₂ is not a Mediator of Caspase-Independent Cell Death

When RBS-HeLa cells were pretreated with 100 μM allopurinol for 1 hour prior to 55 μM SSe for 24 hours to effectively reduce intracellular H₂O₂ levels, DNA fragmentation levels remained unchanged compared to PBS-pretreated controls (FIG. 24). Similar results were seen when RBS-MCF7 cells were pretreated with allopurinol for 1 hour prior to the addition of 55 μM SSe or 1 mM Res for 24 hours (FIGS. 26 and 28). These findings show that in these cases of caspase-independent cell death, H₂O₂ is not a direct mediator of cell death, but rather increases as a consequence of cell death.

Elevated Levels of ROS can be Detected by the RLuc8:fLuc Ratio in Living Subjects

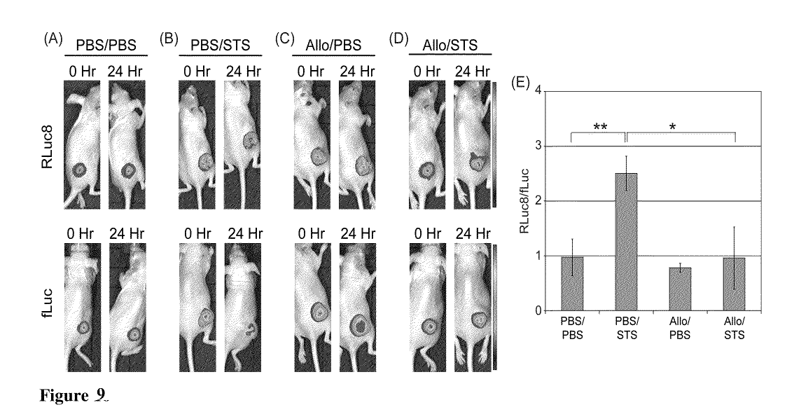

To investigate the capability of the RBS to detect oxidative stress in vivo, HeLa-fR tumor xenografts were grown in nude mice (n=3 per group). Tumors were initially treated with PBS or allopurinol as an inhibitor, followed by PBS or STS 1 hour later. As seen in FIG. 9A, control mice that were treated with PBS as an inhibitor and PBS as treatment (PBS/PBS) exhibited little change in RLuc8 bioluminescence after 24 hours (top panel). Similarly, the fLuc bioluminescence also did not change significantly in PBS/PBS-treated mice (FIG. 9A, bottom panel). In contrast, in PBS/STS-treated mice, the RLuc8 bioluminescence remained stable (FIG. 9B, top panel), but the fLuc bioluminescence was significantly diminished after 24 hours (FIG. 9B, bottom panel). When mice were treated with inhibitor alone (Allo/PBS), the tumors again exhibited little change in RLuc8 bioluminescence (FIG. 9C, top panel), but there was a slight increase in fLuc bioluminescence (FIG. 9C, bottom panel) after 24 hours. Finally, when mice were treated with the inhibitor and the cell death inducer (Allo/STS), tumors showed little change in bioluminescence for RLuc8 (FIG. 9D, top panel) and fLuc (FIG. 9D, bottom panel). The bioluminescence values were quantified for each group of animals and the RLuc8:fLuc ratio values are reported in FIG. 9E. Consistent with the cell culture studies, the ratio increased significantly (p<0.01) between the PBS/PBS and PBS/STS groups. The RLuc8:fLuc ratio decreased slightly from the PBS/PBS groups to the Allo/PBS groups; however, the change was not statistically significant. Nonetheless, this slight change can indicate that tumors possess slightly elevated levels of intracellular ROS even without STS-treatment. A significant reduction in the RLuc8:fLuc ratio (p<0.05) was observed between the PBS/STS and Allo/STS groups. These results provide evidence that ROS are elevated in tumors in response to STS-treatment and that the increased level of oxidative stress is reflected by an increase in the RLuc8:fLuc ratio.

TABLE 7
Reagents, manufacturers and working concentrations used in this study.

| | | | Working | |
|---|---|---|---|---|
| | Reagent | Manufacturer | Concentration | |
| Stress | Staurosporine (STS) | Sigma | 0-50 | μM |
| Inducers | Hydrogen Peroxide (H2O2) | Fisher | 0-10 | mM |
| | Hypoxanthine (HX) | Sigma | 50 | μM |
| | Xanthine Oxidase (XO) | Sigma | 25 | mU/mL |
| Proteasome | MG-132 | Fisher | 20 | μM |
| Inhibitors | Epoxomicin | Enzo Life Sciences | 10 | μM |
| | Lactacystin | Sigma | 10 | μM |
| Protease | Calpain Inhibitor III | Calbiochem | 100 | μM |
| Inhibitors | Pepstatin A | Enzo Life Sciences | 100 | μM |
| | Ammonium Chloride | Alfa Aesar | 1 | mM |
| | z-vad-fmk | Sigma | 1 | μM |
| O₂^{•-} | Tiron | Sigma | 10 | mM |
| scavengers | TEMPOL | Sigma | 10 | mM |
| | MnTMPyp | Enzo Life Sciences | 100 | μM |
| •OH | Mannitol | Sigma | 100 | mM |
| scavengers | DFO | Calbiochem | 50 | μM |
| | TEPA | Sigma | 50 | μM |
| H₂O₂- | Catalase | Sigma | 40 | U/mL |
| related Inhibitors | Allopurinol | MP Biomedicals | 100 | μM |
| | Acetylsylic Acid (Aspirin) | Fisher | 1 | mM |

In this study, we found that fLuc activity was rapidly lost in apoptotic cells, while RLuc8 exhibited significantly prolonged functionality under the same conditions. Inhibition assays that targeted intracellular protein degradation/modification pathways that are often involved in apoptosis revealed that ROS, primarily H₂O₂, were responsible for the rapid loss in fLuc activity. Consistent with these findings, allopurinol, an inhibitor of xanthine oxidase-a primary source of intracellular ROS, aspirin, a COX inhibitor, and catalase, an H₂O₂ scavenger, exhibited a protective effect on fLuc activity in apoptotic cells. Moreover, the direct application of H₂O₂ to purified fLuc proteins also led to dose- and time-dependent reduction in bioluminescent activity and the preferential carbonylation of fLuc, compared with RLuc8.

We have shown here that the bioluminescent ratio RLuc8:fLuc can still be used to relay important information regarding intracellular levels of ROS, in particular hydrogen peroxide. For example, we showed that the increase in the level of intracellular H₂O₂ in various cases of caspase-dependent and caspase-independent cell death was accurately reflected by an increase in the RLuc8:fLuc ratio. These findings show that measurements of RLuc8:fLuc ratio could potentially be used to assist with the identification and evaluation of cancer therapeutics that induce cell death through a wide range of morphologically distinct pathways. They can have applications related to caspase-independent cell death considering the anticancer drug resistance and tumorigenesis have been linked to the ability of certain cancer types to evade caspase activation. Currently, there is a dearth of tools available for the study of caspase-independent cell death. Therefore, having a bioluminescent read-out that can be readily translated from benchtop assays to in vivo disease models is particularly beneficial. With an increasing number of pathologies being linked to oxidative stress, including atherosclerosis, cancer, cystic fibrosis, type-2 diabetes, and Alzheimer's disease, the ability to detect oxidative can allow for advances in anti- and pro-oxidant research across a wide range of disciplines.

### Supplemental Methods

### Cell Culture

Human cervical carcinoma (HeLa) and human breast adenocarcinoma (MCF7) cells (ATCC, Manassas, Va.) were grown in Eagle's Minimum essential medium (Mediatech, Manassas, Va.) supplemented with 10% fetal bovine serum (FBS, HyClone, Logan, Utah), 1.5 g/L sodium bicarbonate, 100 U/mL penicillin and 100 μg/mL streptomycin (Invitrogen). Human embryonic kidney (293T/17) cells (ATCC) were grown in Dulbecco's Modified Eagle's Medium (DMEM, Mediatech) supplemented with 10% FBS (Hyclone), 1.5 g/L sodium bicarbonate, 100 U/mL penicillin and 100 μg/mL streptomycin (Invitrogen). The genetically modified human embryonic kidney cells (293FT, Invitrogen) for generating lentiviral particles were cultured in Dulbecco's Modified Eagle's Medium (DMEM, Mediatech) supplemented with 10% fetal bovine serum (FBS, HyClone, Logan, Utah), 1.5 g/L sodium bicarbonate, 1 mM sodium pyruvate, 0.1 mM MEM non-essential amino acids (NEAA), 6 mM L-glutamine, 100 U/mL penicillin and 100 μg/mL streptomycin (Invitrogen). Cells that were genetically engineered to stably express the fR vector (described below) also had Blasticidin (Invitrogen) added at a final concentration of 4 μg/mL. All cells were cultivated in a 37° C. humidified incubator with 5% CO₂.

### Lentiviral Particle Production and Stable Cell Line Creation

Lentiviral particles containing the fR vector were produced using the Virapower Lentiviral Directional TOPO Expression Kit (Invitrogen) according to the manufacturer's instructions. Briefly, 293FT cells were transfected with viral packaging plasmids and the fR lentiviral vector using Lipofectamine 2000 (Invitrogen). Viral supernatant was harvested 48 hours after transfection, concentrated using Peg-it Virus Concentration solution (System Biosciences, Mountain View, Calif., USA) and the titer was assessed. Concentrated viral particles were added to HeLa cells, which were subsequently selected for stable genomic integration using Blasticidin (Invitrogen), resulting in HeLa-fR cells.

### Cellular Bioluminescence Imaging

HeLa-fR cells were plated at a density of 10,000 cells/well in a black-walled 96 well tissue culture plate (BD Biosciences). The Dual-Glo Luciferase Assay System (Promega) was utilized according to the manufacturer's instructions in order to obtain bioluminescent images in an Omega 16vs imaging system (UltraLum, Claremont, Calif., USA). It should be noted that the exact position of the 96 well plate was kept constant throughout the imaging session in order for accurate ratiometric images to be calculated.

### Microplate Image Analysis

All image analyses were performed using ImageJ (NIH, Bethesda, Md., USA). Image background was determined by measuring 3 regions of interest (ROIs) surrounding the bioluminescent ROI. After background subtraction, the RLuc8 image was divided by the fLuc image using the ‘Math’ command under the ‘Process’ menu tab.

### Proteasome Inhibition Control Assay

HeLa-fR cells were plated at a density of 10,000 cells/well in a white-walled 96-well plate (BD Biosciences). 24 hours later, cells were treated with PBS (pH 7.4), 20 μM MG-132, 10 μM epoxomicin or 10 μM lactacystin for 1 hour. HeLa-fR cells were then treated with PBS (pH 7.4) or 10 μM Staurosporine (STS) in addition to the inhibitors for 24 hours. Proteasome activity measurements were obtained using the Proteasome-Glo 3-Substrate Cell-Based Assay system (Promega) according to the manufacturer's instructions.

### Protease Inhibition Control Assays

Calpain Inhibitor III: HeLa-fR cells were plated at a density of 10,000 cells/well in a white-walled 96-well plate (BD Biosciences). 24 hours later, cells were treated with either PBS (pH 7.4) or 100 μM Calpain Inhibitor III for 1 hour. PBS (pH 7.4) or 10 μM STS was subsequently added to the cells, and both compounds remained on the cells for 24 hours. Calpain activity measurements were obtained using the Calpain-Glo Protease Assay (Promega) according to the manufacturer's instructions.

Pepstatin A: HeLa-fR cells were plated at a density of 10,000 cells/well in a black-walled 96-well plate (BD Biosciences). 24 hours later, cells were treated with either PBS (pH 7.4) or 100 μM pepstatin A for 1 hour, with either PBS (pH 7.4) or 10 μM STS added for another 24 hours. Cathepsin activity was assessed using the CV-Cathepsin B Detection Kit (Enzo Life Sciences) according to the manufacturer's instructions with one exception; after the final wash step, the plate was read on an Infinite 200 plate reader (Tecan) for fluorescence (550ₑₓ/610ₑₘ), as opposed to microscopy.

Ammonium Chloride: In lieu of a control assay for ammonium chloride effectively inhibiting lysosome-phagosome fusion, we direct the reader to a seminal paper regarding this phenomena in HeLa cells (1).

z-vad-fmk: HeLa-fR cells were plated at a density of 10,000 cells/well in a white-walled 96-well plate (BD Biosciences). 24 hours later, cells were treated with either PBS (pH 7.4) or 1 μM z-vad-fmk for 1 hour. PBS (pH 7.4) or 10 μM STS was subsequently added to the cells, and both compounds remained on the cells for 24 hours. Caspase activity measurements were obtained using the Caspase-Glo 3/7 Assay (Promega) according to the manufacturer's instructions.

### Superoxide (O₂^{•-}) Scavenger Control Assay

HeLa-fR cells were plated at a density of 120,000 cells per well of a 12 well tissue culture plate (BD Biosciences). 24 hours later, cells were treated with PBS (pH 7.4), 10 mM TEMPOL, 10 mM Tiron or 100 μM MnTMPyP for 1 hour. PBS (pH 7.4) or 10 μM STS was subsequently added to the cells, and both compounds remained on the cells for 24 hours. Intracellular O₂^{•-} levels were then determined by incubating the cells in 5 μM dihydroethidium (DHE, Invitrogen) for 30 minutes at 37° C. and subjecting them to flow cytometry using a Guava EasyCyte (Guava Technologies, Hayward, Calif., USA). Analysis of flow cytometry data was accomplished using FlowJo software (TreeStar, Ashland, Oreg., USA).

### Hydroxyl Radical (^{•}OH) Scavenger/Inhibitor Control Assay

HeLa-fR cells were plated at a density of 120,000 cells per well of a 12 well tissue culture plate (BD Biosciences). 24 hours later, the cells were incubated with 10 μM hydroxyphenylfluorescein (HPF, Sigma) for 30 minutes at 37° C. Cells were then treated with PBS (pH 7.4), 100 mM Mannitol, 50 μM deferoxamine (DFO), 50 μM tetraethylenepentamine (TEPA) or 50 μM DFO plus 50 μM TEPA for 1 hour. PBS (pH 7.4) or 10 μM STS was subsequently added to the cells, and all compounds remained on the cells for 24 hours. Intracellular ^{•}OH levels were determined by subjecting the cells to flow cytometry using a Guava EasyCyte (Guava Technologies). Analysis of flow cytometry data was accomplished using FlowJo software (TreeStar).

### Hydrogen Peroxide (H₂O₂)-Related Inhibitor Control Assays

Catalase and Allopurinol: HeLa-fR cells were plated at a density of 120,000 cells per well of a 12 well tissue culture plate (BD Biosciences). 24 hours later, the cells were incubated with 10 μM CM-H₂DCFDA (Invitrogen) for 30 minutes at 37° C. Cells were then treated with PBS (pH 7.4), 50 U/mL catalase or 100 μM allopurinol for one hour. PBS (pH 7.4) or 10 μM STS was subsequently added to the cells, and all compounds remained on the cells for 24 hours. Intracellular H₂O₂ levels were determined by subjecting the cells to flow cytometry using a Guava EasyCyte (Guava Technologies). Analysis of flow cytometry data was accomplished using FlowJo software (Treestar).

Aspirin: HeLa-fR cells were plated at a density of 1.2×10⁶ cells in 100 mM tissue culture dishes (BD Biosciences). 24 hours later, the cells were treated with PBS (pH 7.4) or 1 mM aspirin for one hour, followed by PBS (pH 7.4) or 10 μM STS for 24 hours. Cyclooxygenase (COX) activity was determined by using the Cox Activity Assay Kit (Cayman Chemical Company, Ann Arbor, Mich., USA).

### Cell Death Assays

Caspase Activity: PCD was induced (as described above) 24 hours after plating 10,000 cells/well in white-walled 96 well tissue culture plates (BD Biosciences). After indicated treatment times, the Caspase 3/7-Glo Assay (Promega) was performed according to the manufacturer's protocol and bioluminescence measurements were obtained from and Infinite 200 plate reader (Tecan). All values were normalized to controls (i.e. pre-treatment values).

DNA Fragmentation: PCD was induced (as described above) 24 hours after plating 300,000 cells/well in 6 well tissue culture plates (BD Biosciences). After indicated treatment times, TUNEL assays for DNA fragmentation were performed using the TUNEL/ApoBRDU assay kit (Invitrogen) according to the manufacturer's protocol. Percent DNA fragmentation was determined using flow cytometry on a Guava EasyCyte flow cytometer (Guava Technologies, Hayward, Calif.). Analysis of flow cytometry data was performed using FlowJo software (Treestar, Ashland, Oreg.).

### Quantitative Real Time PCR

Cytoplasmic RNA from HeLa-fR cells cultured under indicated conditions was isolated using the High Pure RNA Isolation Kit (Roche, Mannheim, Germany) and subsequently reverse-transcribed to single-stranded cDNA using a High-Capacity cDNA Reverse Transcription Kit (Applied Biosystems, Foster City, Calif., USA) according to each manufacturer's protocol. Quantitative RT-PCR was performed on an ABI PRISM 7300 Sequence detection system using FAM-labeled Taqman primer sets for Rluc8, fLuc and β-actin (as a control) and the Taqman universal PCR Master Mix (Applied Biosystems) according to the manufacturer's protocol.

### Western Blotting

Following indicated culture conditions, HeLa-fR cells were washed 3 times with 1×PBS (pH7.4). Proteins were extracted using RIPA extraction buffer (50 mM Tris HCL, pH 7.4, 1% Triton X-100, 0.25% Na-deoxycholate, 150 mM NaCl, 1 mM EDTA and a Complete Mini Protease Inhibitor Cocktail Tablet (Roche)) at 4° C. for 30 minutes with constant agitation. Total protein concentrations were measured using a BCA assay (Pierce, Rockford, Ill., USA). 30 μg of total protein from each sample were heated to 95° C. in Laemmli Sample buffer containing 2% (v/v) 2-mercaptoethanol (Bio-Rad, Hercules, Calif., USA). After a 5 minute cooling period, the samples were quickly centrifuged and the supernatants were immediately run on a 4-15% Tris-HCl gel (Bio-Rad). Proteins separated by electrophoresis were transferred to nitrocellulose membranes in 1× Transfer Buffer (Bio-Rad) at 15V for 30 minutes. Membranes were blocked in Blocking Buffer for Fluorescent Western Blotting (Rockland Immunochemicals, Gilbertsville, Pa., USA) for 60 minutes. The membranes were incubated with anti-fLuc (Sigma), anti-rLuc (Millipore, Billerica, Mass.) or anti-β-actin (Abcam, Cambridge, Mass.) primary antibodies in blocking buffer overnight. After washing 3 times with TBS-T, the membranes were incubated with either Anti-Mouse IgG Antibody IRDye800 conjugated (fLuc, rLuc) or Anti-Rabbit IgG Antibody IRDye800 conjugated (β-actin) at a 1:10,000 dilution (Rockland Immunochemicals). The fluorescent signal from the membranes was imaged using the Odyssey Infrared Imaging System (Li-Cor Biosciences, Lincoln, Nebr., USA).

### Purified Protein Bioluminescence Assay

The DNA sequences encoding Rluc8 and fLuc were PCR amplified and inserted into the pHAT 10 and pHAT 11 vectors (Clontech) between the BamHI and EcoRI restriction sites. 50 mL of bacterial cultures were inoculated in LB medium containing 50 μg/mL ampicillin overnight. Cultures were expanded and protein expression was induced using IPTG (Fisher Scientific, Pittsburgh, Pa., USA). Clarified cell lysate was applied to a purification column loaded with TALON metal affinity resin (Clontech). The Rluc8 and fLuc proteins were eluted from the column using imidazole and purified on Amicon Ultra Centrifugal Filtration Devices (Millipore, Billerica, Mass.) of appropriate molecular weight cutoffs. Equivalent concentrations of each protein were resuspended in a sample buffer consisting of phosphate buffered saline (PBS, Invitrogen), 1 mg/mL boving serum albumin (BSA, Fisher Scientific) and a Complete Mini protease inhibitor cocktail tablet (Roche). Samples were treated with 0, 1, 5 or 10 mM H₂O₂ for 2 hours. Luminescence measurements were made on an Infinite 200 plate reader (Tecan) following the addition of Dual-Glo reagents (Promega).

### Purified Protein SDS-PAGE

Pure fLuc and RLuc8 proteins were obtained as stated above. Proteins (650 nM each) were added to PBS containing a range of H₂O₂ concentrations (0-10 mM) for up to 120 minutes at 37° C. At indicated times, 10 μL aliquots were removed and heated to 95° C. in Laemmli sample buffer containing 2% (v/v) 2-mercaptoethanol (Bio-Rad). After a 5 minute cooling period the samples were immediately centrifuged and run on a 4-15% Tris-HCl gel (Bio-Rad). Proteins were visualized using the Simply Blue stain (Invitrogen) according to the manufacturer's instructions.

### Protein Carbonylation Detection

To assay for protein carbonylation, the OxyBlot Protein Carbonylation Detection Kit (Millipore) was used according to the manufacturer's instructions. Briefly, 650 nM of pure fLuc and RLuc8 proteins were treated with PBS or 5 mM H₂O₂ for 2 hours, followed by denaturation with SDS (final concentration: 6%). The samples were then treated with 2,4-dinitrophenylhydrazine (DNPH) to derivatize the carbonyl groups in the protein side chains to 2,4-dinitrophenylhydrazone (DNP-hydrazone). The protein samples were run on a 4-15% Tris-HCl gel (Bio-Rad). Proteins separated by electrophoresis were transferred to nitrocellulose membranes in 1× Transfer Buffer (Bio-Rad) at 15V for 30 minutes. The membrane was blocked in blocking/dilution buffer (1% BSA/PBS-T (PBS, pH 7.4, 0.05% Tween-20)) for 1 hour and then incubated with rabbit anti-DNP antibody (1:150 dilution in blocking/dilution buffer) for one hour. After rinsing with PBS-T, the membrane was incubated with Anti-Rabbit IgG Antibody IRDye800 conjugated (β-actin) at a 1:10,000 dilution (Rockland Immunochemicals). The fluorescent signal from the membrane was imaged using the Odyssey Infrared Imaging System (Li-Cor Biosciences, Lincoln, Nebr., USA).

### Animal Imaging

Five week old female nude mice (Charles River Laboratories International LLC, Wilmington, Mass.) were obtained and given free access to food and water. All experiments conformed to animal care protocols approved by the Institutional Animal Care and Use Committee (IACUC) of the University of Pennsylvania. For tumor induction, 2.5×10⁶ HeLa-fR cells were resuspended in PBS (pH 7.4) and subcutaneously implanted in the lower right flank of lightly anesthetized mice (1% isoflurane/oxygen mixture). Once the tumors reached ˜1 cm in diameter, each group was imaged prior to any inhibitor/drug treatment. Briefly, the animals were i.p. injected with 200 μL of sterile 15 mg/mL luciferin (Biosynth, Itasca, Ill.) in PBS (pH 7.4) and imaged using an IVIS Lumina II (Caliper Life Sciences, Hopkinton, Mass.) under anesthesia (2.5% isoflurane/oxygen mixture). The substrate was allowed to clear for 3 hours before the animals received an i.v. injection of 100 μL of 500 μg/mL sterile coelenterazine (NanoLight, Pinetop, Ariz.) in a 20/20/60 mixture of USP grade ethanol/propylene glycol/PBS and imaged using an IVIS Lumina II under anesthesia (2.5% isoflurane/oxygen mixture). Immediately following the second imaging session animals were divided into 4 groups (PBS/PBS, PBS/STS, Allo/PBS, Allo/STS) and the inhibitor (1 mM Allopurinol) or control (PBS, pH 7.4) was intratumorally injected. After 1 hour, the cell death inducer (500 μM STS) or control (PBS, pH 7.4) was intratumorally injected. All intratumoral injections were of a volume of 25 μL. 24 hours later, the animals were imaged in the same manner as stated previously.

### Animal Image Analysis

All image analysis was performed using Living Image Software (Caliper Life Sciences). Images were first background subtracted (treating the animal body as the background) and automatic (5% threshold) ROIs were generated around each tumor. Bioluminescent counts were recorded and used in the ratio calculations. For each group, data was normalized to the day 1 ratio values.

### Example 3

### Optimizing the Design of the Bioluminescent Oxidative Stress Sensor (BOSS)

The inventors have showed that when fLuc and RLuc8 are co-expressed in apoptotic cells the disparity in their loss of activity could provide a unique bioluminescent measurement that is indicative of oxidative stress. This finding shows that the sensitivity of our measurements or the duration can potentially be modulated for specific applications by using alternative strains or genetic variants of luciferase enzymes that exhibit either shorter or longer intracellular lifetimes. For example, a luciferase enzyme that loses activity faster than wild-type fLuc in apoptotic cells is likely to generate a statistically significant detectable change in activity at lower levels of oxidative stress. This would be beneficial for applications where high sensitivity is warranted. Alternatively, if it is necessary to monitor a broad range of oxidative stress levels it may be beneficial to identify a more stable variant of luciferase. Similarly, a luciferase enzyme that is more stable than RLuc8 would provide an important improvement in the dynamic range of oxidative stress measurements as well as increase in the time over which PCD can be detected. Therefore, the lifetime of various strains and genetic variants of the luciferase enzyme in cells exposed to oxidative stress can be examined. The specific enzyme strains that can be tested may include Gaussia (NanoLight), Chroma-Luc CBG68luc (Promega), Chroma-Luc CBRluc (Promega), Metridia (Clontech), and RLuc8.6-535. A genetic variant of firefly luciferase that has previously been shown to improve structural stability can also be constructed. In addition, oxidation-insensitive/sensitive genetic variants of each luciferase strain can be made by mutating various amino acids. For example, methionine, cysteine, tyrosine, tryptophan, histidine and several other amino acids are known to be particularly vulnerable to oxidative damage and thus can either be inserted or removed via mutagenesis to alter the sensitivity of luciferase to ROS or proteolysis.

The pair of optically distinct enzymes that exhibit the greatest disparity in lifetimes in apoptotic cells can be cloned into a lentivirus vector with an intervening IRES site for further investigation. Of particular interest are optically distinct enzymes that utilize the same substrate (i.e. coelenterazine or luciferin), since this can simplify in vitro and in vivo assays. However, if there is a pair of optically distinct enzymes that exhibit a much greater disparity in lifetime than other enzyme pairs but utilize different substrates, they can also be cloned into a lentivirus vector for further analysis. Finally, the pair of enzymes that exhibit similar emission spectrums but utilize different substrates can also be cloned into a lentivirus vector for further analysis. Although this pair of enzymes can not allow for simultaneous imaging of both bioluminescent signals (i.e. sequential administration of substrate would be necessary), the tissue absorption of emitted photons would be similar for both enzymes. Therefore, the bioluminescent ratio should not change as a function of tissue depth, but only the state of oxidative stress within the tumor cells.

Once pairs of luciferase enzymes are identified for incorporation into BOSS, their sensitivity and dynamic range can be evaluated following treatment of genetically engineered cells with STS and the various oxidative agents shown in Table 2. The mechanism responsible for the change in the bioluminescent ratio can be confirmed by performing inhibition studies as described in aim 1. Additional therapeutics can also be tested, including doxorubicin, camptothecin, and TRAIL, in order to provide insight into the versatility of BOSS in detecting caspase-dependent PCD. Further, the luciferase enzymes can be tested in various cell lines.

### Experimental Details:

### Preparation of Luciferase Plasmids:

The luciferase strains Gaussia (NanoLight), Chroma-Luc CBG68luc (Promega), Chroma-Luc CBRluc (Promega), and Metridia (Clontech) can be purchased from their respective vendors. The coding sequence for each enzyme can be amplified by PCR and ligated into pcDNA3.1+ (Invitrogen), which possesses a CMV promoter. The secretion signaling sequence can be removed from the Gaussia and Metridia sequences. RLuc8.6-535 can be prepared by performing multi site-directed mutagenesis (QuickChange Kit; Stratagene) on RLuc8 (A123S, D154M, E155G, D162E, I163L, V185L) according to the manufacturer's instructions. A genetic variant of firefly luciferase (fLuc5) that has previously been shown to improve structural stability will also be constructed. Specifically, five solvent-exposed non-conserved hydrophobic amino acids in Firefly Luciferase can be substituted with five hydrophilic residues, F14R, L35Q, V182K, I232K, and F465R. The peak emission wavelength and substrate for each enzyme is provided in Table 5.

TABLE 5
Luciferase enzymes and their respective peak emission wavelength
and substrate

| | Peak Emission | |
|---|---|---|
| Luciferase Enzyme | Wavelength | Substrate |
| Firefly | 560 nm | luciferin |
| Renilla | 480 nm | coelenterazine |
| Gaussia | 480 nm | coelenterazine |
| Chroma Luc CBG68luc | 537 nm | luciferin |
| Chroma-Luc CBRluc | 613 nm | luciferin |
| Metridia | 480 nm | coelenterazine |
| RLuc8 | 480 nm | coelenterazine |
| RLuc8.6-535 | 535 nm | coelenterazine |
| fLuc5 | 560 nm | luciferin |

The amino acids that are susceptible to specific ROS have been widely reported and this knowledge can be used for decisions about genetic mutations. For example, methionine, cysteine, arginine, lysine, proline and threonine, are all known to be susceptible to hydroxyl radical-mediated damage. If it is determined that the fLuc cleavage is occurring, which seems to be the case, then susceptible amino acids can be mutated within solvent exposed areas. Alternatively, if it is determined that H₂O₂-mediated oxidation leads to signal loss, then amino acids near the fLuc active site can be modified. If it is found that proteases are responsible for the loss in fLuc activity, then solvent exposed areas can be made more hydrophilic. Genetic variants can be made for each of the luciferase strains listed in Table 5, as deemed appropriate.

### Luciferase Stability Measurements:

Human cervical carcinoma cells (Hela, ATCC) can be engineered to stably express each of the luciferase strains and genetic variants. The stable cells will be seeded onto 96-well plates and apoptosis will be induced 24 hours later with 10 mM staurosporine (STS, Sigma-Aldrich) and bioluminescence measurements will be recorded over the course of 24 hours (i.e. 0, 2, 4, 6, 8, 12, and 24 hours) using a Tecan Infinite M200 plate reader. Each measurement can be performed in triplicate on three separate occasions.

The pair of optically distinct enzymes that exhibit the greatest disparity in lifetimes in apoptotic cells can be cloned into a lentivirus vector (pLenti6/V5-D-TOPO, Invitrogen) with an intervening IRES site (IRES2, Invitrogen) for further investigation. Of particular interest are optically distinct enzymes that utilize the same substrate, since this can simplify in vitro and in vivo assays. However, if there is a pair of optically distinct enzymes that exhibit a much greater disparity in lifetime than other enzyme pairs but utilize different substrates, they can also be cloned into a lentivirus vector for further analysis. Finally, the pair of enzymes that exhibit similar emission spectrums but utilize different substrates can be cloned into a lentivirus vector for further analysis.

### Sensitivity and Dynamic Range:

Drug dosing studies can be conducted to determine the minimum extent of oxidative stress and the minimum percent of cells that must be undergoing cell death to be accurately detected by BOSS. Specifically, Hela, 293T, and MDA-MB-231 cells stably expressing each bioluminescent sensor can be seeded onto 96-well plates and bioluminescent activity can be recorded after treatment with various doses of staurosporine (0 to 50 mM) over a time frame of 0 to 24 hours. All measurements will be recorded on a Tecan Sapphire plate reader and the bioluminescent ratio, (activity of stable luciferase):(activity of unstable luciferase), can be calculated. Untreated cells can be used as controls for each time point. Ratiometric measurements can be compared with results from commercially available assays for oxidative stress and cell death. The assay used to measure oxidative stress can depend on the mechanism responsible for the loss in bioluminescent signal. Options for the various pathways of oxidative stress are listed in Table 1. PCD can be measured using the Apo BrDu TUNEL assay (Invitrogen) and Caspase-Glo 3/7 (Promega). The sensitivity of the bioluminescent sensor may be defined as the lowest percent of oxidative stress/cell death that can be accurately measured (i.e. p<0.05, two-tailed t-test, unpaired). The dynamic range may be defined as the maximum achievable bioluminescent ratio. Inhibition studies can also be conducted to confirm the mechanism of oxidative stress leading to a change in the bioluminescent ratio.

Similar oxidative stress and cell death experiments can be conducted with the agents listed in Table 2, as well as doxorubicin, TRAIL, and camptothecin. Multiple cell lines and multiple drugs can be tested to determine the versatility of the probe under a diverse set of experimental conditions.

Alternative Approaches: Chroma Luc CBG68luc and RLuc8.6-535 are the preferred choices for the substrate-distinct pair of enzymes used in BOSS due to their similar optical properties. If both of these enzymes exhibit similar lifetimes in apoptotic cells then it can be tested whether the wild-type, i.e. unstable, form of RLuc can be red-shifted to emit at 535 nm as opposed to the more stable RLuc8, using the same mutations.

Finding an optically distinct pair of enzymes that catalyze the same substrate is not expected to be a problem, since one certain option would be to combine RLuc8.6-535 with either RLuc or RLuc8, depending on the stability of RLuc8.6-535 in apoptotic cells.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A ratiometric bioluminescent sensor for imaging a biological condition comprising:
a first luminescent protein; and
a second luminescent protein,
wherein said first and second luminescent proteins exhibit different characteristics associated with said biological condition, and thereby illuminates differently in response to said biological condition, and
wherein a ratio between the luminescence of said first and said second luminescent proteins indicates a change in said biological condition.

2. The bioluminescent sensor of claim 1, wherein said first and second luminescent proteins have different half life, and wherein said first luminescent protein degrades (or loses activity) at a first time and said second luminescent protein degrades (or loses activity) at a second time in response to said biological condition.

3. The bioluminescent sensor of claim 1, wherein said first luminescent protein catalyzes a first substrate and said second luminescent protein catalyzes a second substrate in response to said biological condition.

4. The bioluminescent sensor of claim 1, wherein said first luminescent protein is a first luciferase protein and said second luminescent protein is a second luciferase protein.

5. The bioluminescent sensor of claim 4, wherein said first luciferase protein is a wild-type firefly luciferase (fLuc).

6. The bioluminescent sensor of claim 4, wherein said first luciferase protein is a Renilla luciferase (RLuc).

7. The bioluminescent sensor of claim 4, wherein said second luciferase protein is RLuc8.

8. The bioluminescent sensor of claim 4, wherein said second luciferase protein is a mutant firefly luciferase.

9. The bioluminescent sensor of claim 8, wherein said mutant firefly luciferase is fLuc5.

10. The bioluminescent sensor of claim 8, wherein said mutant firefly luciferase comprises a mutation F14R, L35Q, V182K, I232K, or F465R.

11. The bioluminescent sensor of claim 4, wherein said first luciferase protein is Gaussia, Chroma Luc CBG68luc, Chroma Luc CBRluc, Metridia, or RLuc8.6-535.

12. The bioluminescent sensor of claim 4, wherein a substrate of said first luciferase protein is luciferin and a substrate of said second luciferase protein is coelenterazine, or vice versa.

13. The bioluminescent sensor of claim 1, wherein said first luminescent protein is operably linked to a gene of interest.

14. The bioluminescent sensor of claim 1, wherein said second luminescent protein is operably linked to a gene of interest.

15. The bioluminescent sensor of claim 1, wherein said biological condition is an oxidative stress.

16. The bioluminescent sensor of claim 1, wherein said first and/or second luminescent proteins exhibit a change in illumination in response to Reactive Oxygen Species (ROS) during an oxidative stress.

17. The bioluminescent sensor of claim 1, wherein said biological condition is an oxidative stress associated disease or condition.

18. The bioluminescent sensor of claim 17, wherein said oxidative stress associated disease is cancer, atherosclerosis, cystic fibrosis, type-2 diabetes, Parkinson's disease, Alzheimer's disease, or an autoimmune disease.

19. The bioluminescent sensor of claim 17, wherein said oxidative stress associated condition is programmed cell death.

20. The bioluminescent sensor of claim 19, wherein said programmed cell death is an apoptotic cell death.

21. The bioluminescent sensor of claim 19, wherein said programmed cell death is a non-apoptotic cell death.

22. The bioluminescent sensor of claim 1, wherein said bioluminescent sensor images said biological condition in vivo.

23. The bioluminescent sensor of claim 1, wherein said bioluminescent sensor images said biological condition ex vivo.

24. The bioluminescent sensor of claim 1, wherein said bioluminescent sensor images said biological condition in vitro.

25. The bioluminescent sensor of claim 1, wherein said bioluminescent sensor images said biological condition in real time.

26. A method for imaging a biological condition comprising:
detecting the luminescence of a first luminescent protein;
detecting the luminescence of a second luminescent protein; and
determining a ratio between the luminescence of said first and second luminescent proteins,
wherein said first and second luminescent proteins exhibit different characteristics associated with said biological condition, and thereby illuminates differently in response to said biological condition, and
wherein the determined ratio between the luminescence of said first and second luminescent proteins indicates a change in said biological condition.

27. The method of claim 26, wherein said first and second luminescent proteins have different half life, and wherein said first luminescent protein degrades (or loses activity) at a first time and said second luminescent protein degrades (or loses activity) at a second time in response to said biological condition.

28. The method of claim 26, wherein said first luminescent protein catalyzes a first substrate and said second luminescent protein catalyzes a second substrate in response to said biological condition.

29. The method of claim 26, wherein said first luminescent protein is a first luciferase protein and said second luminescent protein is a second luciferase protein.

30. The method of claim 29, wherein said first luciferase protein is a wild-type firefly luciferase (fLuc).

31. The method of claim 29, wherein said first luciferase protein is a Renilla luciferase (RLuc).

32. The method of claim 29, wherein said second luciferase protein is RLuc8.

33. The method of claim 29, wherein said second luciferase protein is a mutant firefly luciferase.

34. The method of claim 33, wherein said mutant firefly luciferase is fLuc5.

35. The method of claim 33, wherein said mutant firefly luciferase comprises a mutation F14R, L35Q, V182K, I232K, or F465R.

36. The method of claim 29, wherein said first luciferase protein is Gaussia luciferase, Chroma Luc CBG68luc, Chroma Luc CBRluc, Metridia, or RLuc8.6-535.

37. The method of claim 29, wherein a substrate of said first luciferase protein is luciferin and a substrate of said second luciferase protein is coelenterazine.

38. The method of claim 26, wherein said first luminescent protein is operably linked to a gene of interest.

39. The method of claim 26, wherein said second luminescent protein is operably linked to a gene of interest.

40. The method of claim 26, wherein said biological condition is an oxidative stress.

41. The method of claim 26, wherein said first and/or second luminescent proteins exhibit a change in illumination in response to Reactive Oxygen Species (ROS) during an oxidative stress.

42. The method of claim 26, wherein said biological condition is an oxidative stress associated disease or condition.

43. The method of claim 42, wherein said oxidative stress associated disease is cancer, atherosclerosis, cystic fibrosis, type-2 diabetes, Parkinson's disease, Alzheimer's disease, or an autoimmune disease.

44. The method of claim 42, wherein said oxidative stress associated condition is a programmed cell death.

45. The method of claim 44, wherein said programmed cell death is an apoptotic cell death.

46. The method of claim 44, wherein said programmed cell death is a non-apoptotic cell death.

47. The method of claim 26, wherein said method is performed to image said biological condition in vivo.

48. The method of claim 26, wherein said method is performed to image said biological condition ex vivo.

49. The method of claim 26, wherein said method is performed to image said biological condition in vitro.

50. The method of claim 1, wherein said method is performed to image said biological condition in real time.

51. The method of claim 1, wherein said change is a progression of said biological condition.

52. The method of claim 51, wherein said change is a progression of a cell death.

53. A method for diagnosis of a biological condition comprising:
detecting the luminescence of a first luminescent protein;
detecting the luminescence of a second luminescent protein; and
determining a ratio between the luminescence of said first and second luminescent proteins,
wherein said first and second luminescent proteins exhibit different characteristics associated with said biological condition, and thereby illuminates differently in response to said biological condition, and
wherein the determined ratio between the luminescence of said first and second luminescent proteins indicates a change in said biological condition.

54. A method for monitoring the progression of a biological condition comprising:
detecting the luminescence of a first luminescent protein;
detecting the luminescence of a second luminescent protein; and
determining a ratio between the luminescence of said first and second luminescent proteins,
wherein said first and second luminescent proteins exhibit different characteristics associated with said biological condition, and thereby illuminates differently in response to said biological condition, and
wherein the determined ratio between the luminescence of said first and second luminescent proteins indicates the progression of said biological condition.

55. A method for imaging an oxidative stress comprising:
detecting the luminescence of a first luminescent protein;
detecting the luminescence of a second luminescent protein; and
determining a ratio between the luminescence of said first and second luminescent proteins,
wherein said first and second luminescent proteins exhibit different characteristics associated with said oxidative stress, and thereby illuminates differently in response to said oxidative stress, and
wherein the determined ratio between the luminescence of said first and second luminescent proteins indicates a change of said oxidative stress.

56. A method for imaging a programmed cell death comprising:
detecting the luminescence of a first luminescent protein;
detecting the luminescence of a second luminescent protein; and
determining a ratio between the luminescence of said first and second luminescent proteins,
wherein said first and second luminescent proteins exhibit different characteristics associated with said programmed cell death, and thereby illuminates differently in response to said programmed cell death, and
wherein the determined ratio between the luminescence of said first and second luminescent proteins indicates the progression of said cell death.

57. A method for screening drugs to treat a biological condition, said method comprising:
detecting the luminescence of a first luminescent protein;
detecting the luminescence of a second luminescent protein; and
determining a ratio between the luminescence of said first and second luminescent proteins,
wherein said first and second luminescent proteins exhibit different characteristics associated with said biological condition, and thereby illuminates differently in response to said biological condition, and
wherein the determined ratio between the luminescence of said first and second luminescent proteins indicates a change of said biological condition, thereby screening drugs to treat said biological condition.

58. The method of claim 57, wherein said screening is performed in vivo on a subject.

59. The method of claim 58, wherein said subject is a model animal.

60. The method of claim 57, wherein said screening is performed ex vivo.

61. The method of claim 57, wherein said screening is performed in vitro.

62. The method of claim 26, wherein said first and second luminescent protein catalyze the same substrate type in response to said biological condition.

63. The method of claim 28 or 62, wherein said first and second luminescent protein have unique optical properties.

64. The method of claim 29, wherein said first and second luciferase protein catalyze the same substrate type in response to said biological condition.

65. The method of claim 37 or 64, wherein said first and second luminescent protein have unique optical properties.
